(19) United States
(12) Patent Application Publication
(10) Pub. No.: US 2025/0042925 A1
(43) Pub. Date: Feb. 6, 2025

(54) Improved Compositions and Methods for Targeting Mitochondria in Cancer Cells

(71) Applicant: The Medical College of Wisconsin, Inc., Milwaukee, WI (US)

(72) Inventors: Michael B. Dwinell, Elm Grove, WI (US); Mahmoud O. AbuEid, Deerfield, IL (US); Brian Christopher Smith, Elm Grove, WI (US); Robert Frederick Keyes, Pleasant Prairie, WI (US)

(21) Appl. No.: 18/715,433

(22) PCT Filed: Dec. 2, 2022
(86) PCT No.: PCT/US2022/051665

§ 371 Date: May 31, 2024

Related U.S. Application Data
(60) Provisional application No. 63/285,374, filed on Dec. 2, 2021.

(51) Int. Cl. C07F 9/54; A61K 31/662; A61P 35/00

(52) U.S. Cl. CPC C07F 9/5442; A61K 31/662; A61P 35/00

(57) ABSTRACT

The present disclosure provides compounds of formula (I′) and uses thereof for the treatment of cancer. Also provided are pharmaceutical compositions and kits comprising the same compounds. The present compounds may have improved anti-tumor effect and reduced toxicity and may be useful for targeting mitochondria in cancer cells.

### CROSS-REFERENCE TO RELATED APPLICATIONS

The present application claims priority to U.S. Provisional Patent Application No. 63/285,374, filed Dec. 2, 2021, the content of which is hereby incorporated by reference in its entirety.

### STATEMENT REGARDING FEDERALLY SPONSORED RESEARCH OR DEVELOPMENT

Not Applicable.

### BACKGROUND

Metformin, a biguanide from Galega officinalis, is an FDA-approved drug for treating diabetes, which inhibits hepatic gluconeogenesis. Metformin exists as a hydrophilic cation at physiological pH and targets mitochondria, albeit rather inefficiently. Metformin has been in use in the clinic for over 50 years and has a very good safety profile (diabetic patients tolerate daily doses of 2-3 grams). Previous work has shown that increasing mitochondrial targeting of metformin (mito-metformin) enhances its antiproliferative effects in cancer cells.

There remains a need for compounds that have improved properties, including potency, selectivity, and toxicity, that are effective in inhibiting tumor formation (e.g., reducing the severity or slowing the progression of symptoms of cancer).

### SUMMARY OF THE INVENTION

Disclosed herein are modified mito-metformin compounds, pharmaceutical compositions comprising the same, and uses of the compounds and pharmaceutical compositions for treating cancer in a subject in need thereof.

In one aspect, the present disclosure provides a compound of formula (I′),

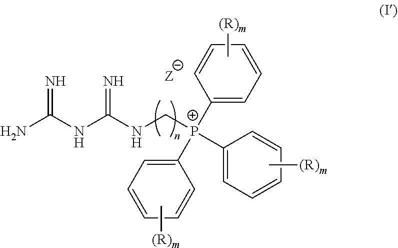

wherein
R at each occurrence is independently F, Cl, CH₂X, CHX₂, CX₃, or OCH₃;
m at each occurrence is independently 1, 2, 3, 4, or 5;
n is an integer from 1-20;
X at each occurrence is independently halogen; and
Z is a counterion.

In another aspect, the present disclosure provides a pharmaceutical composition. The pharmaceutical composition comprises the compound as disclosed herein and a pharmaceutically acceptable carrier, diluent, or excipient.

In a further aspect, the disclosure provides a method of treating cancer in a subject in need thereof. The method comprises administering a therapeutically effective amount of the compound as described herein or the pharmaceutical composition as described herein to the subject in order to treat the cancer.

In another aspect, the disclosure provides a method of reducing or inhibiting cancer cell growth in a subject in need thereof. The method comprises administering a therapeutically effective amount of the compound as described herein or the pharmaceutical composition as described herein to the subject to reduce or inhibit cell growth.

In another aspect, the disclosure provides a method of inhibiting, reducing or treating metastasis of a cancer in a subject in need thereof. The method comprises administering to the subject a therapeutically effective amount of the compound as described herein or the pharmaceutical composition as described herein to the subject in need thereof to inhibit, reduce or treat the metastasis.

In yet another aspect, the disclosure provides use of a compound as described herein for the manufacture of a medicament for the treatment of cancer.

In yet another aspect, the disclosure provides a kit. The kit comprises the pharmaceutical composition as described herein and instructional material.

### BRIEF DESCRIPTION OF THE DRAWINGS

FIG. 1 demonstrates the impact of aryl ring substitutions on electron density and lipophilicity of MMe. Chemical structures and electron density maps of MMe (b) and the analogs pMeO-MMe (a), mCF₃-MMe (c), and pCF₃-MMe (d). The heatmap depicts the range of electron density for all four compounds. Red indicates increased electron density (decreased partial positive charge); blue indicates decreased electron density (increased partial positive charge). The arrows and white and black markers denote the range of electron density seen for MMe.

FIGS. 2A-2E demonstrate the growth inhibitory effects of MMe, pCF₃-MMe, and mCF₃-MMe on pancreatic, ovarian, breast, and colon cancers and non-transformed normal epithelial cells. Logarithmic dose-response fit curves of KPC1242, Panc-1, AsPC-1, HPSC, and HPNE confluence in response to treatment with MMe (FIG. 2A) or pCF₃-MMe (FIG. 2C), or OVCAR-8, OVCAR-4, MDA-MB-231, and HCT-116 confluence in response to treatment with MMe (FIG. 2B) or pCF₃-MMe (FIG. 2D). Densitometric quantification (FIG. 2E) in KPC1242 cells treated with MMe (0.7 μM) or pCF₃-MMe (3 μM). Representative images of levels of Ki-67 (green) were recorded using the IncuCyte of KPC1242 treated with MMe (0.7 μM) or pCF₃-MMe (3 μM), followed by quantification of the total integrated intensity of Ki-67. Values are mean±SD, n=3-4, each experiment completed in technical duplicates or triplicates. IC₅₀ values calculated from confluence curves are shown in FIGS. 7A-7H, FIGS. 8A-8H, FIGS. 9A-9J, FIGS. 10A-10C, FIGS. 11A-11I. Statistical differences in e were determined using Student's t-test (*, p<0.05; **, p<0.01).

FIGS. 3A-3H show pCF₃-MMe inhibits complex I derived mitochondrial respiration. FIGS. 3A and 3E show representative mitochondrial stress tests on the KPC1242 pancreatic cancer cell line preceded by 24-hour treatment with MMe (FIG. 3A) or pCF₃-MMe (FIG. 3E). Injections of oligomycin (1 μg/mL), 2,4-dinitrophenol (50 μM), rotenone (1 μM), and antimycin A (10 μM) were injected as indicated by the arrows. FIGS. 3B and 3F show representative complex I derived oxygen consumption (OCR) test after 24-hour treatment of increasing concentrations of MMe (FIG. 3B) or pCF₃-MMe (FIG. 3F) followed by rotenone to inhibit complex I and succinate to bypass complex I inhibition. FIGS. 3C and 3G show logarithmic dose-response curves of basal oxygen consumption rate in response to increasing concentrations of MMe (c) or pCF₃-MMe (g) in KPC1242 cells. FIGS. 3D and 3H show logarithmic dose-response curves of complex I derived oxygen consumption rate in response to increasing concentrations of MMe (FIG. 3D) or pCF₃-MMe (FIG. 3H) in KPC1242 cells. Treated cells were normalized to vehicle (FIGS. 3C, 3G, and 3D). Values are mean±SD from 3 biological replicates completed in technical quadruplicate.

FIGS. 4A-4G demonstrate pCF₃-MMe selectively depolarizes mitochondria and activates bioenergetic signaling in cancer cells. AsPC-1 and HPSC cells were serum-starved overnight, loaded with tetramethylrhodamine ethyl ester (TMRE), and treated for 1 hour with the indicated concentrations of MMe or pCF₃-MMe or, as the vehicle control, DMSO. Mitochondrial TMRE retention was measured by flow cytometry. Representative histograms of n=3 of TMRE fluorescence in AsPC-1 (FIG. 4A) and HPSC (FIG. 4B) in response to treatment. Normalized Mean fluorescence intensity of TMRE in treated AsPC-1 (FIG. 4C) shows the depolarization of mitochondrial membrane in response to treatment and a lack of depolarization in the HPSC cells (FIG. 4D). Total cellular ATP levels were analyzed using a luminescent ATP detection assay of KPC1242 cells treated with MMe (0.7 μM) or pCF₃-MMe (3 μM), and changes in ATP levels were assayed (FIG. 4E). Representatives immunoblot analyses of phosphorylated and total AMPK levels (FIG. 4F) and densitometric quantification (FIG. 4G) in KPC1242 cells treated with MMe (0.7 μM) or pCF₃-MMe (3 μM). Representative images of levels of Ki-67 (green) were recorded using the IncuCyte of KPC1242 treated with MMe (0.7 μM) or pCF₃-MMe (3 μM), followed by quantification of the total integrated intensity of Ki-67. Values are mean±SD from 3 biological replicates. Statistical differences in a, c, and e were determined using Student's t-test (*, p<0.05; **, p<0.01; ***, p<0.001, ns: not significant).

FIGS. 5A-5C shows quantification of pCF₃-MMe persistence within the tumor microenvironment. Tumor engraftment and treatment schedule. KPC1242 murine pancreatic cancer engrafted mice were treated with 1 mg pCF₃-MMe (FIG. 5A). Following treatment, pCF₃-MMe was extracted using 8:2 MeOH:H₂O at 30 minutes, 2 hours, 4 hours, 6 hours, or 24 hours and levels of pCF₃-MMe at each time point were quantified using quantitative NMR (FIG. 5B). Nonlinear fit curve analysis of pCF₃-MMe levels was used to generate t½. Values are mean±SEM from 1-4 biological replicates (FIG. 5C).

FIGS. 6A-6I show pCF₃-MMe inhibits tumor growth with enhanced biosafety relative to MMe. C57BL/6J mice were treated with 1 mg MMe, 1 mg pCF₃-MMe, 1 mg MMe, 250 μg MMe, 100 μg MMe, 50 μg MMe, or 25 μg MMe for 6 hours (FIG. 6A) or 5 days (FIG. 6D). Kaplan-Meier survival curve of mice treated for 6 hours (FIG. 6B) or 5 days (FIG. 5E), n=3-5. Numbers of mice surviving over time after a single injection (FIG. 6C) or 5 repeated doses (FIG. 6F). Significance was determined using a Mantel-Cox test (*, p<0.05; **, p<0.01). KPC1242 murine pancreatic cancer engrafted mice were treated with 1 mg pCF₃-MMe or vehicle 5 days on and 2 days off (FIG. 6G). Tumors isolated from vehicle and treated mice were pictured (FIG. 6H). Tumor volume was measured ex vivo from vehicle and pCF₃-MMe-treated mice (FIG. 6I). Values are mean±SD. Significant differences were determined by Student's t-test (*, p<0.05).

FIGS. 7A-7H demonstrate effects of MMe on pancreatic, ovarian, breast, and colon cancers cell lines. Daily confluence measured as a surrogate for cell proliferation of KPC1242 (FIG. 7A), Panc-1 (FIG. 7B), AsPC-1 (FIG. 7C), HCT-116 (FIG. 7D), MDA-MB-231 (FIG. 7E), OVCAR-4 (FIG. 7F), and OVCAR-8 (FIG. 7G) cells treated with increasing concentrations of MMe (0.1 μM-10 μM). Representative phase-contrast images at day 5 in vehicle or MMe-treated Panc-1 and HCT-116 cell lines (green) overlaid by the confluence mask (FIG. 7H).

FIGS. 8A-8H demonstrate effects of pCF₃-MMe on pancreatic, ovarian, breast, and colon cancers cell lines. Daily confluence of KPC1242 (FIG. 8A), Panc-1 (FIG. 8B), AsPC-1 (FIG. 8C), HCT-116 (FIG. 8D), MDA-MB-231 (FIG. 8E), OVCAR-4 (FIG. 8F), and OVCAR-8 (FIG. 8G) cells treated with increasing concentrations of pCF₃-MMe (0.1 μM-10 μM). Representative phase-contrast images at day 5 in vehicle or MMe-treated Panc-1 and HCT-116 cells (green) overlaid by the IncuCyte confluence mask (FIG. 8H).

FIGS. 9A-9J show effects of mCF₃-MMe on pancreatic, ovarian, breast, and colon cancers cell lines. Logarithmic dose-response fit curves of KPC1242, Panc-1, AsPC-1, HPSC, and HPNE cell confluence in response to treatment with mCF₃-MMe (FIG. 9A), or OVCAR-8, OVCAR-4, MDA-MB-231, and HCT-116 cell confluence in response to treatment with mCF₃-MMe (FIG. 9B). Daily confluence measured as a surrogate for cell proliferation of KPC1242 (FIG. 9C), Panc-1 (FIG. 9D), AsPC-1 (FIG. 9E), HCT-116 (FIG. 9F), MDA-MB-231 (FIG. 9G), OVCAR-4 (FIG. 9H), and OVCAR-8 (FIG. 9I) cells treated with increasing concentrations of mCF₃-MMe (0.1 μM-10 μM). Representative phase-contrast images at day 5 in vehicle or MMe-treated Panc-1 and HCT-116 cells (green) overlaid by the confluence mask (FIG. 9J).

FIGS. 10A-10C show effects of pMeO-MMe on a pancreatic cancer cell line. Daily confluence was measured as a surrogate for cell proliferation of pMeO-MMe treated Panc-1 cells (FIG. 10A). Representative phase-contrast images at day 5 in vehicle or pMeO-MMe treated Panc-1 cell line (green) overlaid by the confluence mask (FIG. 10B). Logarithmic dose-response fit curves of Panc-1 cell line confluence in response to treatment with pMeO-MMe (FIG. 10C). Values are mean±SD, n=4, each experiment completed in technical quadruplicate.

FIGS. 11A-11I show effects of MMe, pCF₃-MMe, and mCF₃-MMe on non-malignant cells. Daily confluence was measured as a surrogate for cell proliferation of MMe-treated HPNE (FIG. 11A) and HPSC-1 (FIG. 11B) cell lines. Representative phase-contrast images at day 5 in vehicle or MMe-treated HPNE cell line (green) overlaid by the confluence mask (FIG. 11C). Daily confluence measurement of pCF₃-MMe treated HPNE (FIG. 11D) and HPSC-1 (FIG. 11E) cell lines. Representative phase-contrast images at day 5 in vehicle or pCF₃-MMe-treated HPNE cell line (green) overlaid by the confluence mask (FIG. 11F). Daily confluence measurement of pCF₃-MMe-treated HPNE (FIG. 11G) and HPSC-1 (FIG. 11H) cell lines. Representative phase-contrast images at day 5 in vehicle or pCF₃-MMe-treated HPNE cell line (green) overlaid by the confluence mask (FIG. 11I).

FIGS. 12A-12E show ¹⁹F-NMR detection of pCF₃-MMe in vitro. KPC1242 cells were treated with 1 mg pCF₃-MMe or vehicle (FIG. 12A). Following treatment pCF₃-MMe was extracted using 8:2 MeOH:H₂O (FIG. 12B), 9:1 CHCl₃:MeOH (FIG. 12C), CH₂Cl₂ (FIG. 12D), or 1:1 MeCN:H₂O (FIG. 12E). ¹⁹F peaks are indicated by downwards arrows: pCF₃-MMe signal=−61.6 ppm; plasticizer signal=−73.4 ppm.

FIGS. 13A-13E demonstrate ¹⁹F-NMR detection of pCF₃-MMe in tumors. FIG. 13A shows tumor engraftment and treatment. KPC1242 murine pancreatic cancer engrafted mice were treated with 1 mg pCF₃-MMe. Following treatment pCF₃-MMe was extracted from tumors using 8:2 MeOH:H₂O (FIG. 13B), 9:1 CHCl₃:MeOH (FIG. 13C), CH₂Cl₂ (FIG. 13D), or 1:1 MeCN:H₂O (e). ¹⁹F peaks are indicated by downwards arrows: pCF₃-MMe signal=−61.6 ppm; plasticizer signal=−73.4 ppm.

FIG. 14 shows pCF₃-MMe was not detected in the liver or blood. Tumor engraftment and treatment. KPC1242 murine pancreatic cancer engrafted mice were treated with 1 mg pCF₃-MMe after injection, and the levels of pCF₃-MMe were assayed in the liver and blood from two independent mice.

### DETAILED DESCRIPTION OF THE INVENTION

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

As used in this specification and the claims, the singular forms “a,” “an,” and “the” include plural forms unless the context clearly dictates otherwise. For example, the term “a compound” should be interpreted to mean “one or more compounds” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### Compounds

Increases in the mitochondrial membrane potential are a recognized characteristic in multiple types of solid and liquid cancers. It remains unclear if changes in membrane potential are causal events or secondary events in cancer progression. The molecular mechanisms underlying mitochondrial membrane hyperpolarization remain enigmatic but have been experimentally exploited to selectively target cancer cells instead of normal cells. For example, rhodamine¹²³, mitochondria-penetrating peptides, Szeto-Schiller peptides, and triphenylphosphonium cation (TPP⁺)-conjugated compounds have all been synthesized to selectively target cancer cells. To date, TPP⁺-conjugated compounds have demonstrated the most promising in vitro and in vivo data. TPP⁺-conjugated compounds consist of the mitochondria-targeting TPP⁺ moiety, a linker typically consisting of an aliphatic carbon chain, and a functional cargo. TPP⁺-conjugated compounds were used to uncover the mechanism of coupling oxidative phosphorylation to mitochondrial membrane potential and, subsequently, to understand the role of oxidative metabolism in cancer. Moreover, early efforts were reported which focused on using the antioxidant Mito-Q, a TPP⁺-conjugated synthetic analog of coenzyme Q10, ubiquinone, to understand the role of mitochondrial redox recycling in disease progression. For over a decade, TPP⁺-conjugated compounds have been employed as tumor-selective anti-cancer agents, designing and testing mitochondria-targeted-3-carboxy-proxyl nitroxide, mitochondria-targeted-magnolol, mitochondria-targeted-metformin (MMe) and others as anti-cancer agents. Each demonstrated broad anti-tumor efficacy by inhibiting complex I of the electron transport chain and activating energy-sensing signaling pathways. These TPP⁺-conjugated mitochondria-targeted compounds are examples of the functional consequences of changing the cargo. Changes in the carbon chain linker may skew TPP⁺-conjugated compounds to target complex III over complex I. While previous efforts have focused on modifying the aliphatic linker or the cargo of TPP⁺-conjugated compounds, very few studies have investigated the functional and biological consequences of modifying the TPP⁺ moiety.

The conventional wisdom underlying the use of TPP⁺ was that the large ionic radius, diffuse positive charge, and lipophilic nature allowed conjugated compounds to cross lipid bilayers and accumulate in the more negative inside of mitochondria. Most structural modifications to TPP⁺-conjugated agents have focused on modifying the length of the linker connecting TPP⁺ to cargo to fine-tune lipophilicity and localization of the mitochondria-targeted compound. A recent report showed that TPP⁺ modifications can be employed to dissociate mitochondrial localization from functional effects on mitochondrial respiration and demonstrated a strong role for fluorine modifications in enhancing mitochondrial localization (Kulkarni, C. A. et al. A Novel Triphenylphosphonium Carrier to Target Mitochondria without Uncoupling Oxidative Phosphorylation. J Med Chem. 64, 662-676). Fluorine has different physical and chemical properties from other elements, including bond length, van der Waals radius, van der Waals volume, and electronegativity. Fluorine's small van der Waals radius, 1.46 Å, can easily sterically replace hydrogen with a van der Waals radius of 1.2 Å. Unlike other halogens, non-polarizable fluorine does not participate in halogen bonding. The introduction of fluorine also allows fine-tuning of the electrostatic properties of the compound based on the number and regiochemical placement of the fluorine atoms. The ¹⁹F isotope has 100% natural abundance and a +½-spin, resulting in an easily detectable NMR-active nucleus. As fluorinated compounds are scarce in nature, the introduction of ¹⁹F to a compound enables the use of ¹⁹F NMR detection of fluorinated TPP⁺-conjugated compounds with minimal noise resulting from endogenous molecules. Thus, fluorine substitution may have the added benefit of increasing the detectability of mitochondria-targeted compounds using ¹⁹F NMR or ¹⁸F positron emission tomography.

The present disclosure demonstrates that substituting electron withdrawing groups such as fluorine or chlorine groups on the phosphine's aryl groups of mito-metformin compounds may chemically modify the triphenylphosphonium cation to improve selectivity, lipophilicity, hydrophobicity, efficacy, and detectability of such compounds and reduce their toxicity.

The present disclosure provides improved mito-metformin (MMe) compounds modified to selectively inhibit cancer proliferation and progression with improved stability and decreased side effects. In one aspect, the present disclosure provides a compound of formula (I′)

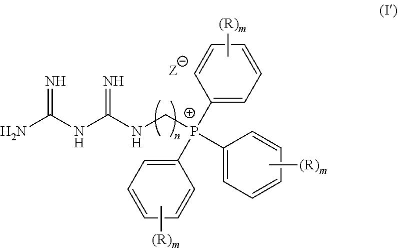

wherein
R at each occurrence is independently F, Cl, CH₂X, CHX₂, CX₃, or OCH₃;
m at each occurrence is independently 1, 2, 3, 4, or 5;
n is an integer from 1-20;
X at each occurrence is independently halogen; and
Z is a counterion.

In some embodiments, the compound of formula (I′) as described herein is a compound of formula (I)

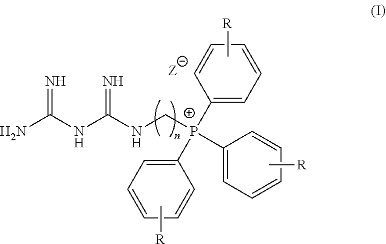

wherein
R is F, Cl, CH₂X, CHX₂, or CX₃; and
X is F or Cl.

If substituents are described as being “independently” selected from a group, each substituent is selected independent of the other. Each substituent, therefore, may be identical to or different from the other substituent(s).

The term “halogen” means a chlorine, bromine, iodine, or fluorine atom. In some embodiments, X is F or Cl. In some embodiments, X is F.

The number “m” represents a total number of R groups on a phenyl group, and m can be any one of 1, 2, 3, 4, or 5. In some embodiments, m is 1. In some embodiments, m is 5.

In some embodiments, R is CX₃. In some embodiments, R is CF₃. In some embodiments, R is CX₃ (such as CF₃) and m is 1. In some embodiments, R is CX₃ (such as CF₃) and m is 5.

In some embodiments,

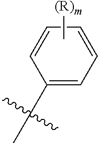

is

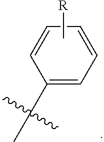

In some embodiments, is

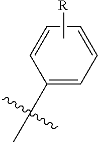

is

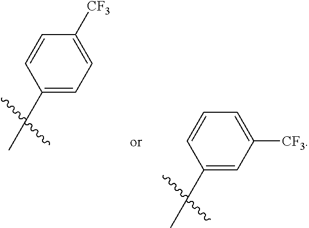 [SMILES: *c1ccc(C(F)(F)F)cc1 ; *c1cccc(C(F)(F)F)c1]

In some embodiments, n is 6, 8, 10, 12, or 14. In some embodiments, n is 10.

Z may be selected from, for example, F⁻, Cl⁻, Br⁻, I⁻, and other suitable negatively charged ions. In some embodiments, Z is Br⁻.

In some embodiments, the compound is

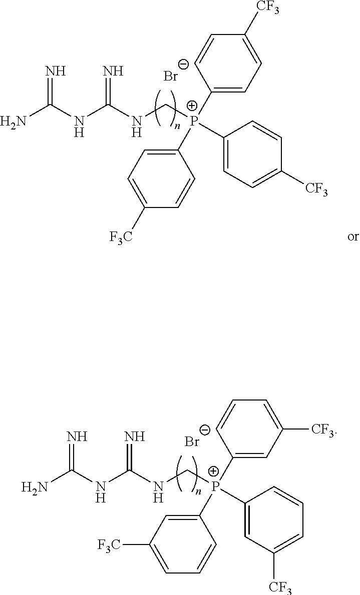

In some embodiments, n is 10 in the compounds described above.

In some embodiments, the compound is

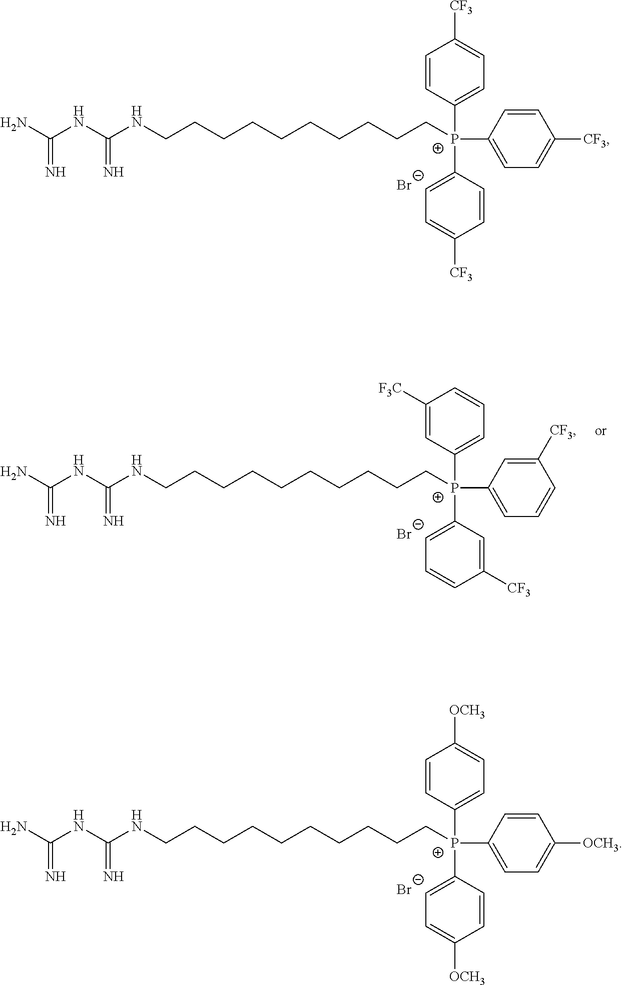 [SMILES: N=C(N)NC(=N)NCCCCCCCCCC[P+](c1ccc(C(F)(F)F)cc1)(c1ccc(C(F)(F)F)cc1)c1ccc(C(F)(F)F)cc1 ; [Br-] ; N=C(N)NC(=N)NCCCCCCCCCC[P+](c1cccc(C(F)(F)F)c1)(c1cccc(C(F)(F)F)c1)c1cccc(C(F)(F)F)c1 ; [Br-] ; COc1ccc([P+](CCCCCCCCCCNC(=N)NC(=N)N)(c2ccc(OC)cc2)c2ccc(OC)cc2)cc1 ; [Br-]]

In some embodiments, the compound is

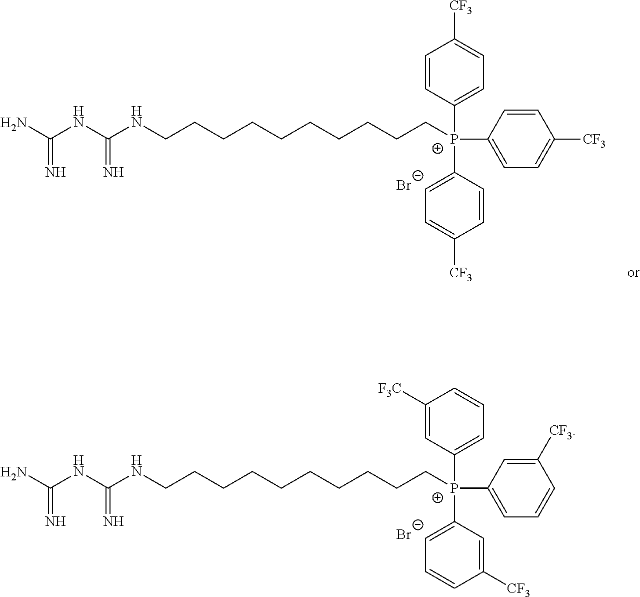 [SMILES: N=C(N)NC(=N)NCCCCCCCCCC[P+](c1ccc(C(F)(F)F)cc1)(c1ccc(C(F)(F)F)cc1)c1ccc(C(F)(F)F)cc1 ; [Br-] ; N=C(N)NC(=N)NCCCCCCCCCC[P+](c1cccc(C(F)(F)F)c1)(c1cccc(C(F)(F)F)c1)c1cccc(C(F)(F)F)c1 ; [Br-]]

### Pharmaceutical Compositions

Another aspect of the disclosure provides a pharmaceutical composition. The pharmaceutical composition comprises the compound as described herein and a pharmaceutically acceptable carrier, diluent, or excipient.

The pharmaceutical composition may include the compound in a range of about 0.1 to 2000 mg. In some embodiments, the pharmaceutical composition may include the compound in a range of from about 0.5 to 500 mg. In some embodiments, the pharmaceutical composition may include the compound in a range of from about 1 to 100 mg. The pharmaceutical composition may be administered to provide the compound at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM).

The compounds may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Optionally, the disclosed compounds or pharmaceutical compositions comprising the disclosed compounds may be administered with additional therapeutic agents, optionally in combination, in order to treat cancers. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical compositions are formulated to comprise the disclosed compounds and further to comprise one or more additional therapeutic agents, for example, one or more additional therapeutic agents for treating cancers.

### Methods of Use

Another aspect of the disclosure provides a method of treating cancer in a subject in need thereof. The method comprises administering a therapeutically effective amount of the compound as disclosed herein or the pharmaceutical composition as disclosed herein to the subject in order to treat the cancer. In one embodiment, the composition comprises one compound as disclosed herein, but in alternate embodiments, multiple compounds as disclosed herein may be administered.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as endpoints any of these disclosed doses (e.g., 2.5 mg-200 mg).

In some embodiments, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as endpoints any of these disclosed dose levels (e.g., 500-2000 ng/kg body weight of the subject).

As used herein, the term “therapeutically effective amount” refers to an amount effective, at dosages and for periods of time necessary, to achieve the desired therapeutic result, such as reduction or inhibition of cell growth in the case of cancers. A therapeutically effective amount of the compounds as disclosed herein may vary according to factors such as the disease state, age, sex, and weight of the subject, and the ability of the disclosed compounds to elicit a desired response in the subject. Dosage regimens may be adjusted to provide the optimum therapeutic response. A therapeutically effective amount is also one in which any toxic or detrimental effects of the compounds as disclosed herein are reduced as compared with known compounds and are outweighed by the therapeutically beneficial effects.

As used herein, the term “tumor” or “cancer” refers to any abnormal proliferation of tissues, including solid and non-solid tumors. For instance, the composition and methods of the present disclosure can be utilized to treat cancers that manifest solid tumors such as pancreatic cancer, breast cancer, colon cancer, lung cancer, prostate cancer, thyroid cancer, ovarian cancer, skin cancer, and the like. The composition and methods of the present disclosure can also be utilized to treat non-solid tumor cancers such as non-Hodgkin's lymphoma, leukemia and the like.

As used herein, the term “subject” refers mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, fish and the like. The term “subject” does not denote a particular age or sex. Preferably, the subject is a human, particularly a human having cancer.

As used herein, the term “treat” or “treating” refers to the management and care of a subject for the purpose of combating the disease, condition, or disorder. Treating includes the administration of a compound of the present disclosure to inhibit, ameliorate and/or improve the onset of the symptoms or complications, alleviating the symptoms or complications, or eliminating the disease, condition, or disorder.

As used here, the term “ameliorate”, “amelioration”, “improvement,” “reduce,” or the like refers to a detectable improvement or a detectable change consistent with improvement occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range about between any two of these values. Such improvement or change may be observed in treated subjects as compared to subjects not treated with the mito-met compounds of the present disclosure, where the untreated subjects have, or are subject to developing, the same or similar disease, condition, symptom or the like. Amelioration of a disease, condition, symptom or assay parameter may be determined subjectively or objectively, e.g., self-assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or by detection of cell migration within a subject. Amelioration may be transient, prolonged or permanent or it may be variable at relevant times during or after the mito-met compounds of the present disclosure is administered to a subject or is used in an assay or other method described herein or a cited reference, e.g., within about 1 hour of the administration or use of the mito-met compounds of the present disclosure to about 3, 6, 9 months or more after a subject(s) has received the compounds as disclosed herein.

As used herein, the term “modulation” (of, e.g., a symptom, level or biological activity of a molecule, replication of a pathogen, cellular response, cellular activity or the like) refers to that the cell level or activity is detectably increased or decreased. Such increase or decrease may be observed in treated subjects as compared to subjects not treated with the mito-met compounds of the present disclosure, where the untreated subjects have, or are subject to developing, the same or similar disease, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or about within any range about between any two of these values. Modulation may be determined subjectively or objectively, e.g., by the subject's self-assessment, by a clinician's assessment or by conducting an appropriate assay or measurement, including, e.g., quality of life assessments or suitable assays for the level or activity of molecules, cells or cell migration within a subject. Modulation may be transient, prolonged or permanent or it may be variable at relevant times during or after the mito-met compounds of the present disclosure is administered to a subject or is used in an assay or other method described herein or a cited reference, e.g., within about 1 hour of the administration or use of the mito-met compounds of the present disclosure to about 3, 6, 9 months or more after a subject(s) has received the compounds as disclosed herein.

As used herein, the term “administering” refers to any means for introducing the compounds as disclosed herein into the body, preferably into the systemic circulation. Examples include but are not limited to oral, buccal, sublingual, pulmonary, transdermal, transmucosal, as well as subcutaneous, intraperitoneal, intravenous, and intramuscular injection.

In some embodiments, the compound used in the method of treating cancer has a reduced toxicity towards a non-transformed cell, as compared with

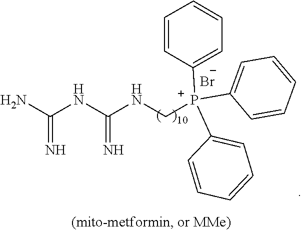

In some embodiments, the compound used in the method of treating cancer is at least one time less toxic, at least two times less toxic, at least three times less toxic, at least four times less toxic, at least five times less toxic, at least six times less toxic, at least seven times less toxic, at least eight times less toxic, at least nine times less toxic, or at least ten times less toxic towards a non-transformed cell, as compared with MMe.

As used herein, the term “toxicity” refers to the degree to which one or more compounds can damage a cell, a group of cells, an organ, or an organism. The “toxicity” of a compound as described herein may be evaluated, for example, in terms of the rate of cell proliferation (e.g., confluence %) or the ability to produce ATP (e.g., extracellular acidification rate, oxygen consumption rate, or % ATP produced) after the cell is contacted by the compound. In general, for two compounds of the same amount, one compound is less toxic to a cell than the other if the cell proliferation rate (confluence %) is higher after the cell is contacted by the compound. Toxicity may be demonstrated by reduction of energy production (such as decreased oxygen consumption rate) induced by the compound. In some embodiments, for two compounds of the same amount, one compound is less toxic to a cell than the other if the extracellular acidification rate of the cell is lower after the cell is contacted by the compound. In some embodiments, for two compounds of the same amount, one compound is less toxic to a cell than the other if the oxygen consumption rate of the cell is higher after the cell is contacted by the compound. In some embodiments, for two compounds of the same amount, one compound is less toxic to a cell than the other if the ATP level in the cell (% ATP) is higher after the cell is contacted by the compound.

As used herein the term “non-transformed cell” refers to cells that are characterized by the absence of any malignant properties.

In some embodiments, the compound used in the method of treating cancer has a higher selectivity towards a cancer cell than a non-transformed cell, as compared with MMe.

In some embodiments, the compound used in the method of treating cancer is at least one time more selective, at least two times more selective, at least three times more selective, at least four times more selective, at least five times more selective, at least six times more selective, at least seven times more selective, at least eight times more selective, at least nine times more selective, or at least ten times more selective towards a cancer cell, as compared with MME.

As used herein, the term “selectivity” refers to the degree to which a substance acts on a cancer cell relative to non-cancer cells. The “selectivity” of a substance, as disclosed herein, is evaluated by comparing the proliferation rate of a cancer cell and that of a normal cell after each cell is contacted by the substance, with a lower proliferation rate of a cancer cell than a normal cell indicating a higher selectivity for the cancer cell.

In some embodiments, the compound selectively inhibits mitochondrial energy metabolism in a cancer cell, relative to a non-transformed cell.

As used herein, the term “mitochondrial energy metabolism” refers to the mitochondrial function of a cell, which may be assessed by measuring the levels of mitochondrial respiration, basal respiration, ATP-linked respiration, maximal and reserve capacities, and/or non-mitochondrial respiration.

The present disclosure demonstrates that the compounds as described herein potently inhibit tumor cell proliferation and induce cytotoxicity by selectively inhibiting tumor, but not normal, cells. In addition, the compounds as described herein are more effective at much lower doses than the doses required with conventional treatments using metformin.

In some embodiments, the compounds as described herein can be combined with ionizing radiation to inhibit tumor cell formation.

In some embodiments, the compounds as described herein can be combined with immune therapies to inhibit tumor cell formation. Suitable immune therapies can include, but are not limited to, immune checkpoint blockade therapy, cellular therapy, and other immune therapies known in the art. Immune checkpoint blockade therapy may involve blocking immune checkpoint proteins produced by immune system cells, such as T cells, from binding with partner proteins on other cells, such as tumor cells. In cellular therapy, viable cells may be injected, grafted, or implanted into a patient to replace or repair damaged tissue and/or cells.

In some embodiments, the compounds as disclosed herein can, when combined with conventional treatment protocols, increase the effectiveness of conventional cancer treatments.

Another aspect of the disclosure provides a method of reducing or inhibiting cancer cell growth in a subject in need thereof. The method comprises administering a therapeutically effective amount of the compound as disclosed herein or the pharmaceutical composition as disclosed herein to the subject to reduce or inhibit cell growth.

In some embodiments, the compound as disclosed herein or the pharmaceutical composition as disclosed herein is administered orally.

Another aspect of the disclosure provides a method of inhibiting, reducing or treating metastasis of a cancer in a subject in need thereof. The method comprises administering to the subject a therapeutically effective amount of the compound as disclosed herein or the pharmaceutical composition as disclosed herein to the subject in need thereof to inhibit, reduce or treat the metastasis.

As used herein, the term “metastasis” refers to the development of secondary malignant growths at a distance from a primary site of cancer.

Another aspect of the disclosure provides use of a compound as disclosed herein for the manufacture of a medicament for the treatment of cancer.

In some embodiments, the cancer in the method as disclosed herein or the use as disclosed herein is breast cancer, colon cancer, gastric cancer, kidney cancer, liver cancer, lung cancer, ovarian cancer, pancreatic cancer, prostate cancer, non-Hodgkin's lymphoma, leukemia, or a combination thereof.

It is surprisingly found that the compounds as disclosed herein have potently increased anti-tumor effect on cell proliferation.

Treating includes the administration of a compound as disclosed herein to reduce, inhibit, ameliorate and/or improve the onset of the symptoms or complications, alleviating the symptoms or complications of the cancer, or eliminating the cancer. Specifically, treatment results in the reduction in tumor load or volume in the patient, and in some instances, leads to regression and elimination of the tumor or tumor cells. As used herein, the term “treatment” is not necessarily meant to imply cure or complete abolition of the tumor. Treatment may refer to the inhibiting or slowing of the progression of the tumor, reducing the incidence of tumor, reducing metastasis of the tumor, or preventing additional tumor growth. In some embodiments, treatment results in complete regression of the tumor. In preferred embodiments, the compound is used to treat pancreatic cancer.

In some embodiments, methods of increasing the anti-tumor response of mito-metformin to a cancer are provided. The method includes the treatment of the cancer with at least one compound as disclosed herein, wherein the compound as disclosed herein results in an increased anti-cancer response. The anti-cancer response, in some embodiments, is characterized by a reduction in the proliferation of cancer cells.

### Kits

Another aspect of the disclosure provides a kit comprising a pharmaceutical composition comprising the compounds as disclosed herein and instructional material.

The term “instructional material” refers to a publication, a recording, a diagram, or any other medium of expression which is used to communicate the usefulness of the present pharmaceutical composition for one of the purposes set forth herein in a human. The instructional material can also, for example, describe an appropriate dose of the present pharmaceutical composition. The instructional material of the present kit can, for example, be affixed to a container which contains a pharmaceutical composition as disclosed herein or be shipped together with a container which contains the pharmaceutical composition. Alternatively, the instructional material can be shipped separately from the container with the intention that the instructional material and the pharmaceutical composition be used cooperatively by the recipient.

### EXAMPLES

The following examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### Example 1. Fluorinated Triphenylphosphonium Analogs Improve Cell Selectivity and In Vivo Detection of Mito-Metformin

Triphenylphosphonium (TPP⁺) conjugated compounds selectively target cancer cells by exploiting their hyperpolarized mitochondrial membrane potential. To date, studies have focused on modifying either the linker or the cargo of TPP⁺-conjugated compounds. Here, we investigated the biological effects of direct modification to TPP⁺ to improve efficacy and detection of Mito-Metformin (MMe), a TPP⁺-conjugated probe we have shown to have promising preclinical efficacy against solid cancer cells. We designed, synthesized, and tested trifluoromethyl and methoxy MMe analogues (pCF₃-MMe, mCF₃-MMe, and pMeO-MMe) against multiple distinct human cancer cells. pCF₃-MMe showed enhanced selectivity towards cancer cells compared to MMe, while retaining the same signaling mechanism. Importantly, pCF₃-MMe allowed quantitative monitoring of cellular accumulation via ¹⁹F-NMR in vitro and in vivo. Furthermore, adding trifluoromethyl groups to TPP⁺ reduced toxicity in vivo while retaining anti-tumor efficacy, opening an avenue to de-risk these next-generation TPP⁺-conjugated compounds.

We rationally redesigned and tested analogues of MMe, selected for its promising preclinical results, by adding electron-withdrawing trifluoromethyl groups in the para and meta positions of the TPP⁺ aryl rings (pCF₃-MMe and mCF₃-MMe). These analogs have reduced electron density on the phosphorus atom and increased lipophilicity at the TPP group, which in turn improve the cell-type selectivity for cancer over normal cells. Importantly, fluorinating the TPP⁺ rings had the added benefit of in vivo detection and quantification via ¹⁹F-NMR. We also synthesized an electron-rich MMe analogue containing para-methoxy aryl rings, pMeO-MMe, that allowed direct comparison of the effect of electron-withdrawing and electron-donating groups on the electron density of the phosphorus atom and the subsequent biological effects of TPP⁺-conjugated compounds. These data are the first to demonstrate that addition of trifluoromethyl groups to the TPP⁺ moiety enhances selectivity for cancer cells, allows reproducible measurement of the accumulation of TPP⁺-conjugated compounds within tumor and non-tumor tissues, and improves the overall safety profile of TPP⁺-conjugated compounds while maintaining potent anti-tumor efficacy in vitro and in vivo.

### Results

### Design of Next-Generation Mitochondria-Targeted Agents by Direct TPP Modification

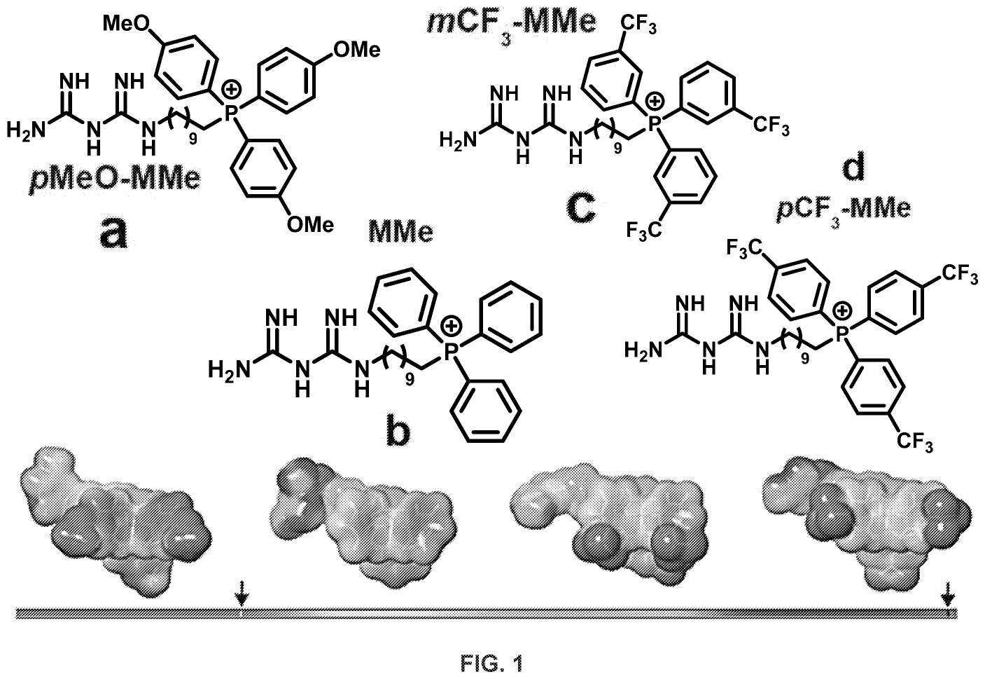

Cationic lipophilic functional groups such as TPP⁺ move across biological membranes, particularly the mitochondrial membrane, without transporters. This phenomenon was previously exploited to selectively deliver metformin into cancer cells to enhance its antitumor efficacy and mitigate potential off-tumor side effects. Here, we the partial positive charge on the phosphorus center of MMe was fine-tuned in a stepwise fashion by adding substituents to the aryl rings of the TPP⁺ moiety. Strong electron-withdrawing trifluoromethyl substituents were added to the meta or para positions of the TPP rings, mCF₃-MMe and pCF₃-MMe, respectively (Scheme 1). Trifluoromethyl substitution at the meta position increases the positive charge on the phosphorus center by withdrawing electrons through the aryl rings inductively. Trifluoromethyl substitution at the para position withdraws electrons both inductively and through resonance effects. Thus, the synthesized molecules were used to test the impact of gradients of increasing partial positive charge on the phosphorus center and lipophilicity of the TPP⁺ moiety for enhanced accumulation in the mitochondria (FIG. 1). By comparison, the partial positive charge at the phosphorus center of MMe was decreased by adding para-methoxy substituents to the TPP aryl rings affording pMeO-MMe (Scheme 1). Methoxy substituents at the para position of the phenyl rings increase electron density throughout the conjugated 7-system via resonance, resulting in decreased partial positive charge at the phosphorus center (FIG. 1). Ultimately, these three novel MMe analogues allowed for a controlled decrease in electron density on the phosphorus atom: pMeO-MMe>MMe>>mCF₃-MMe>pCF₃-MMe (FIG. 1). Likewise, fine-tuning the TPP⁺ aryl rings with trifluoromethyl substituents increased the lipophilicity while methoxy decreased lipophilicity as measured by c Log P (FIG. 1). With these molecules in hand, the biological effects produced by varying the cationic character of the phosphorus center were compared to the parent MMe compound.

Modifying TPP⁺ Increases Selectivity while Maintaining Antitumor Efficacy

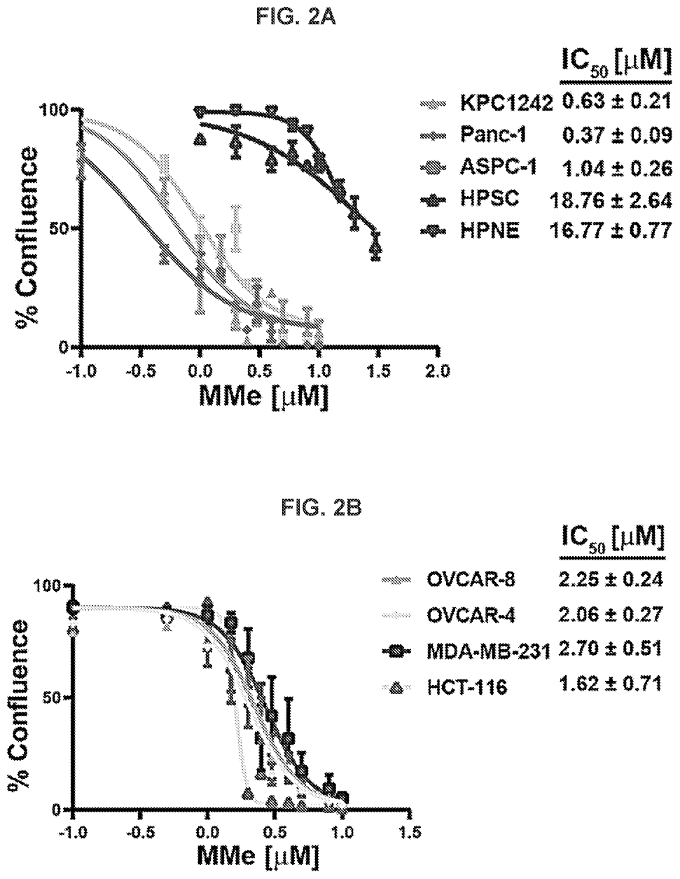

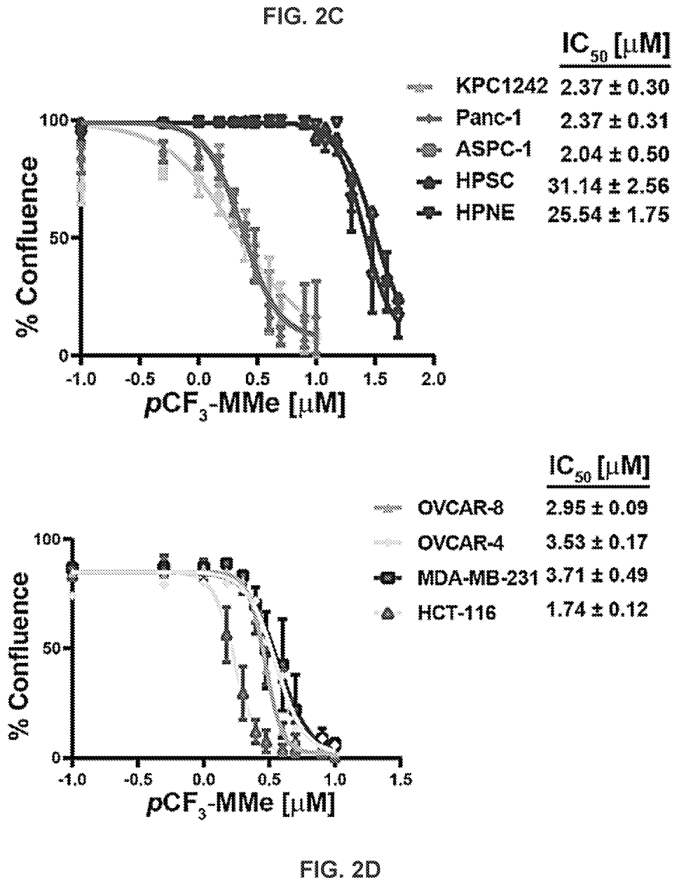

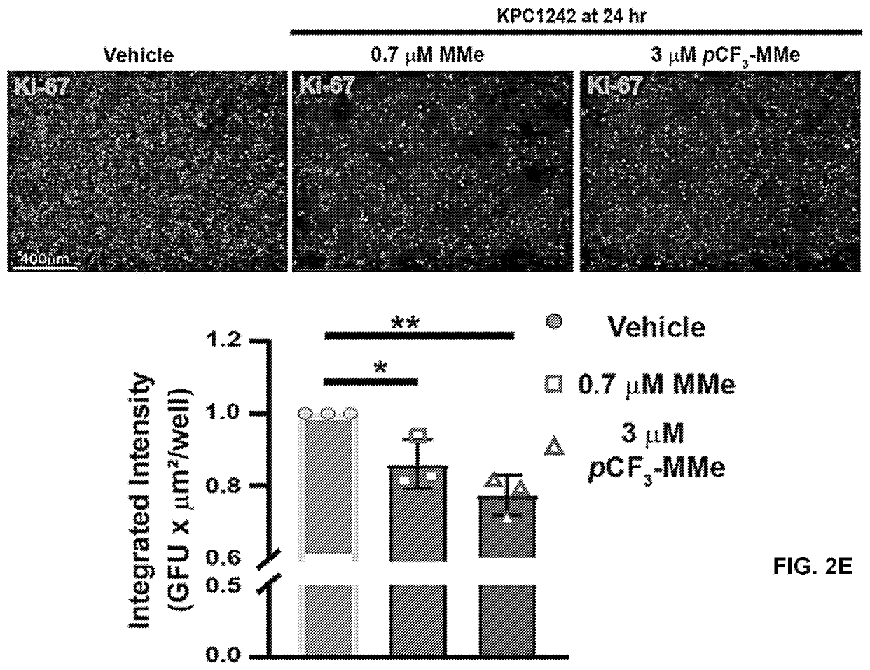

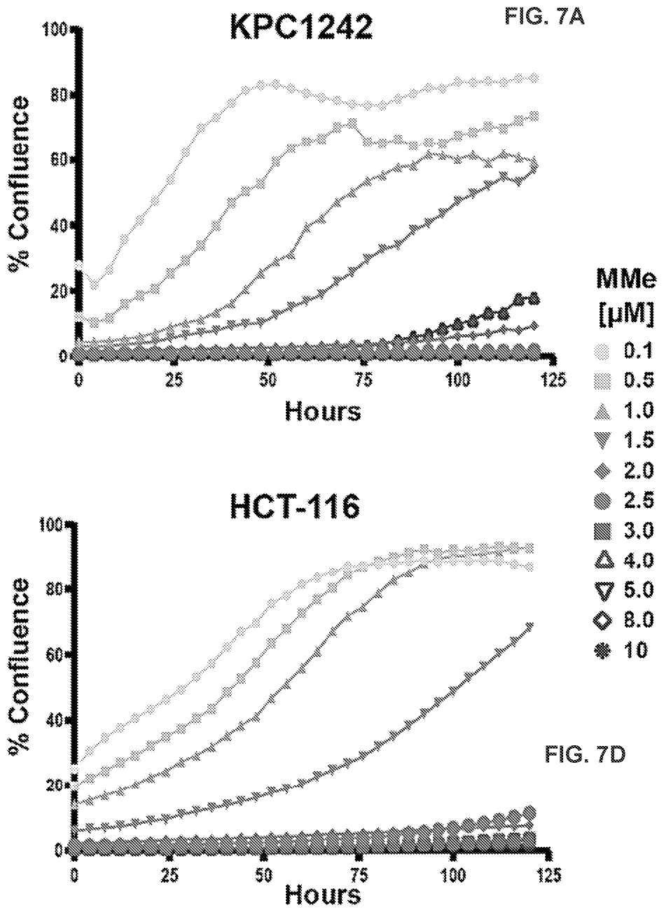

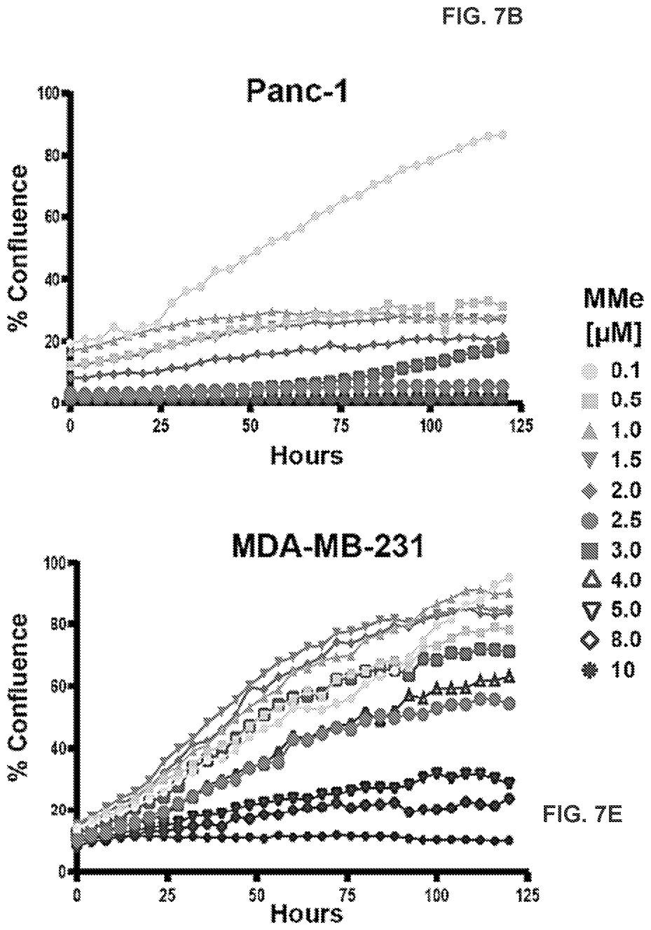

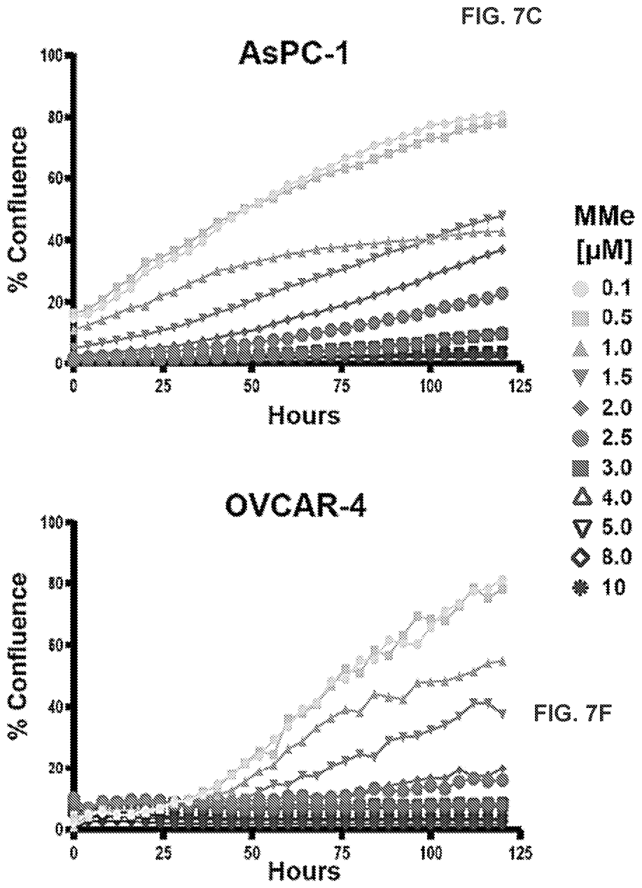

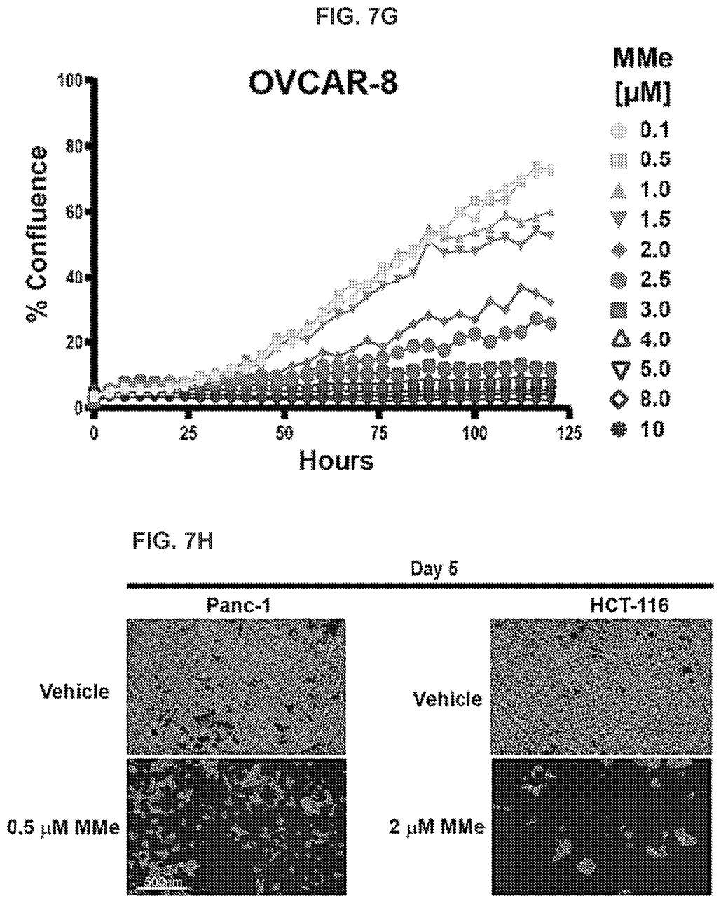

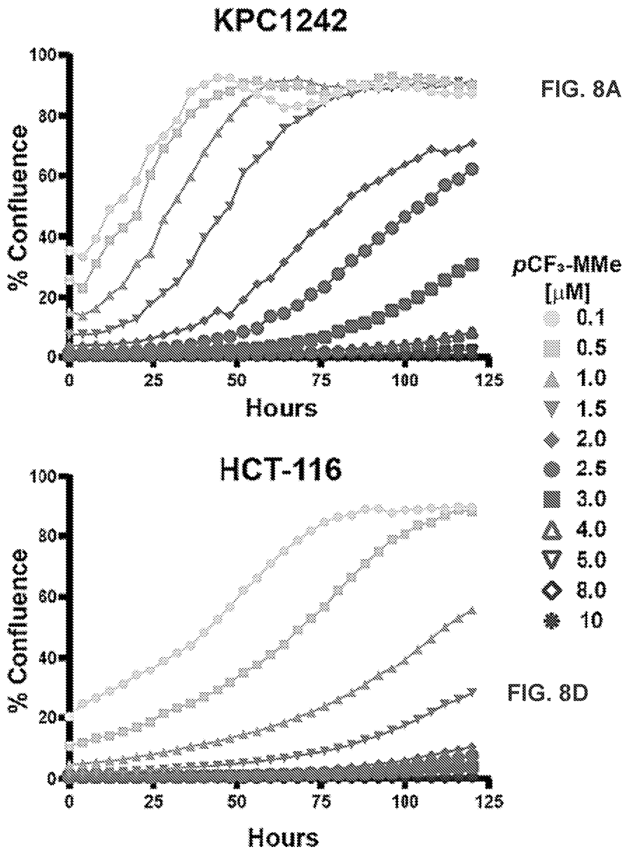

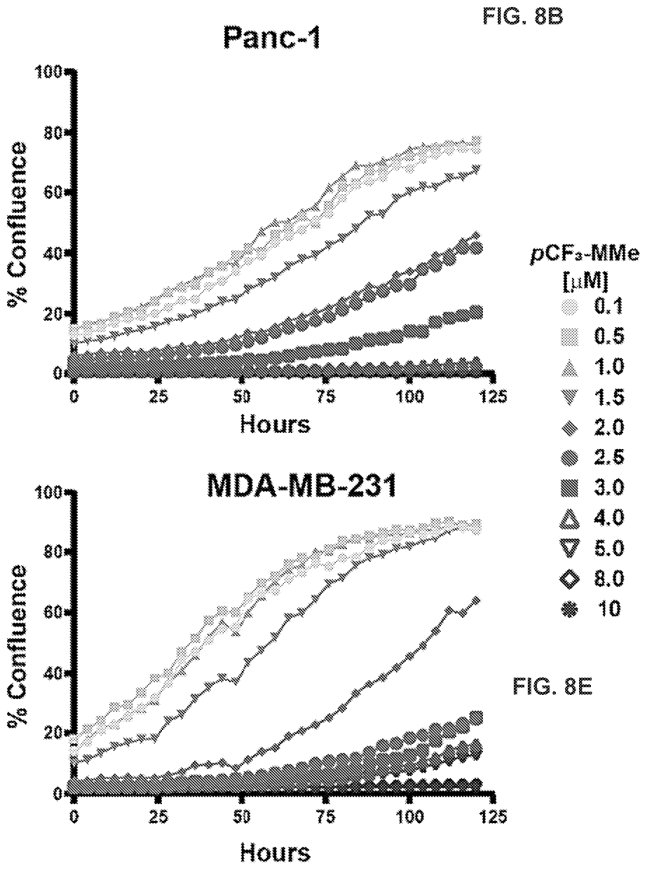

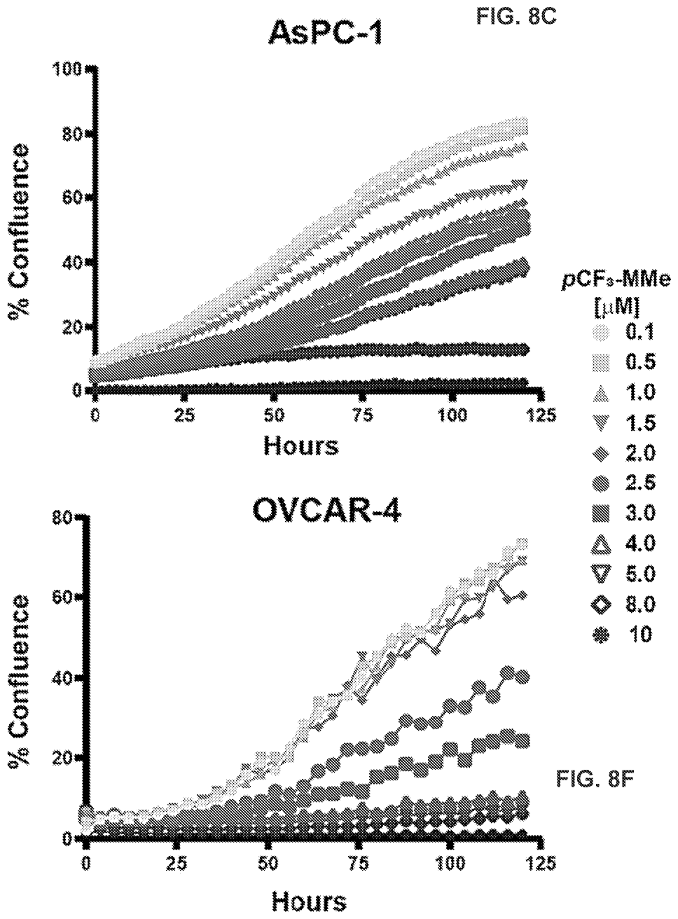

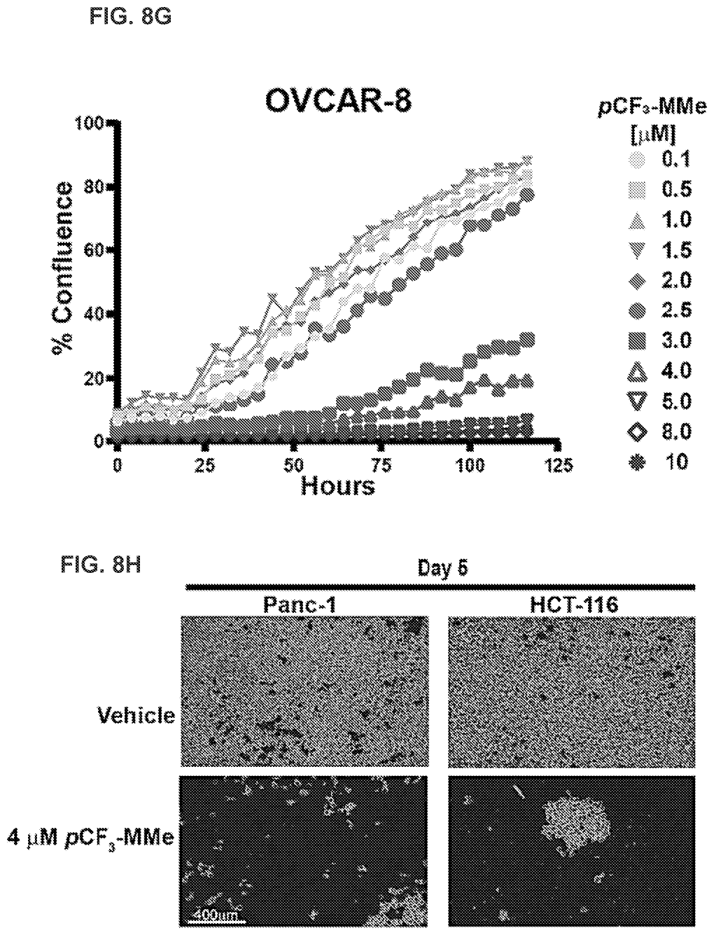

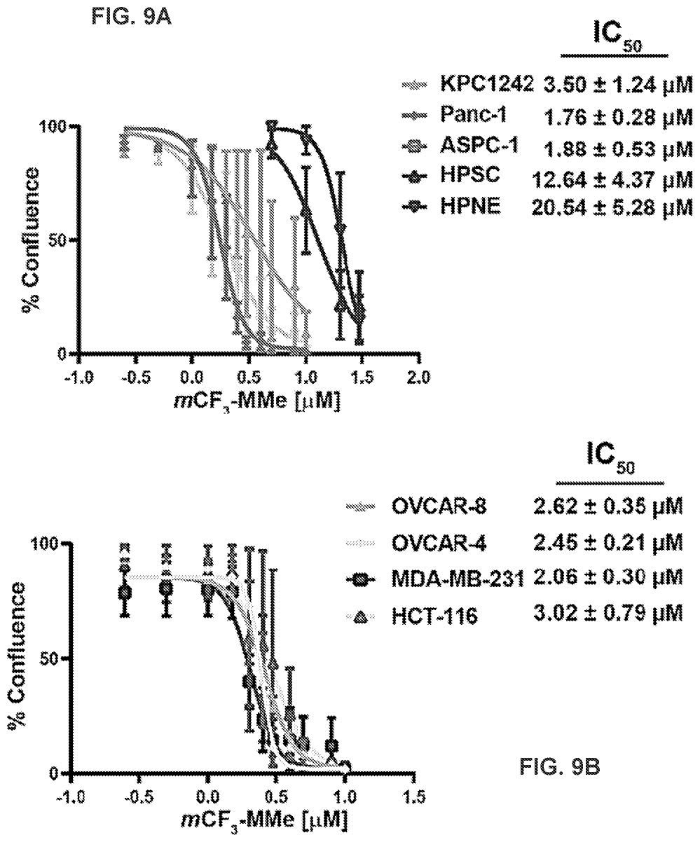

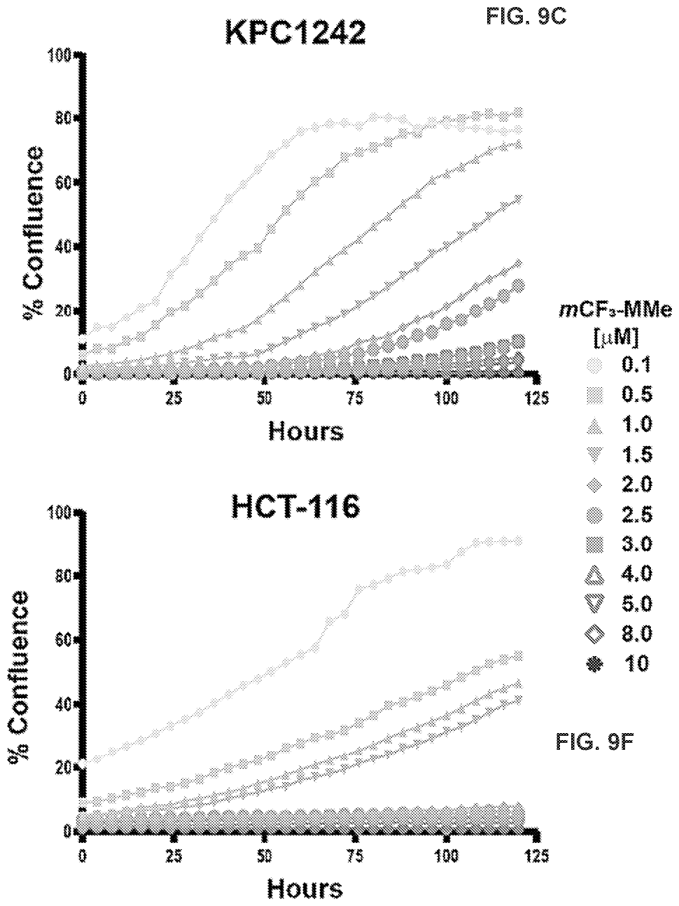

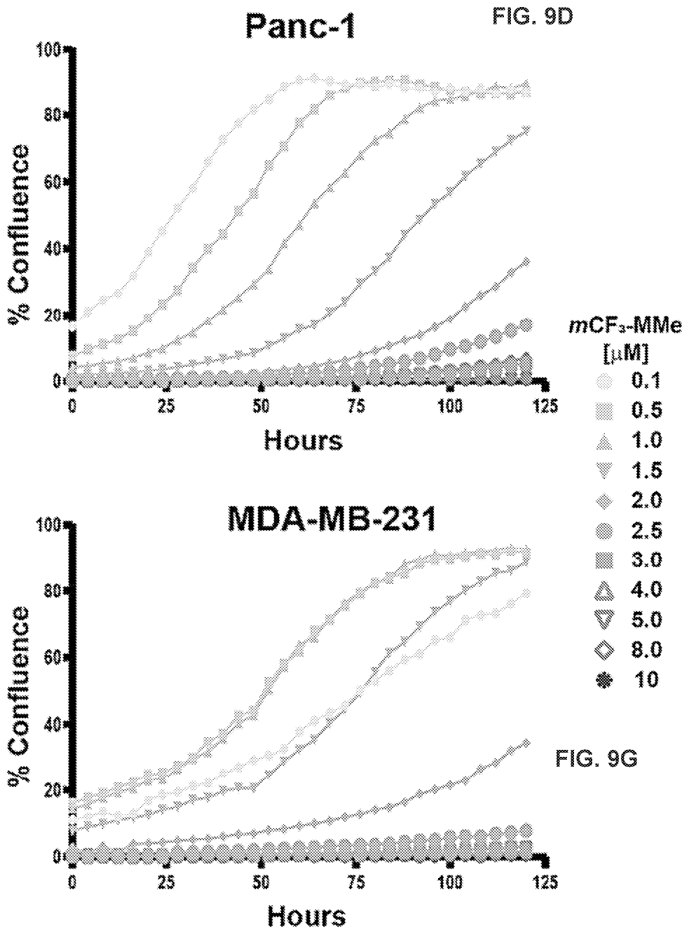

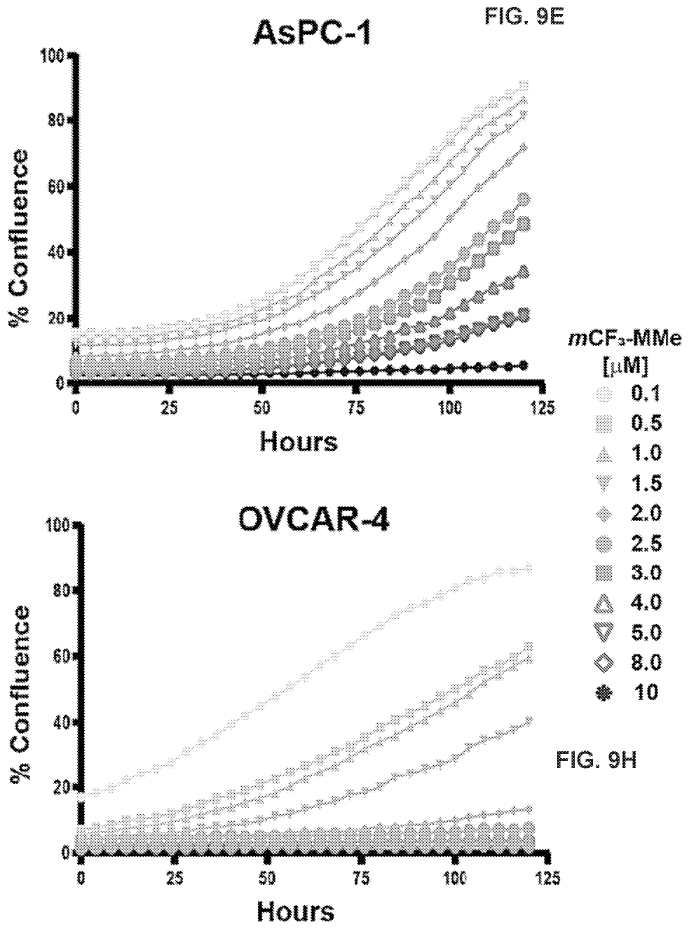

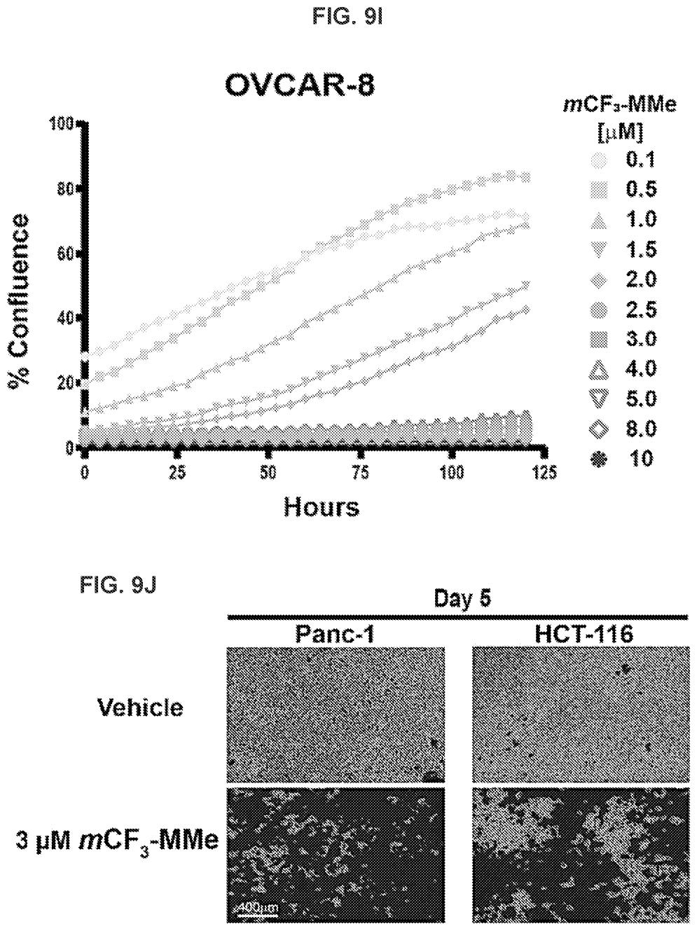

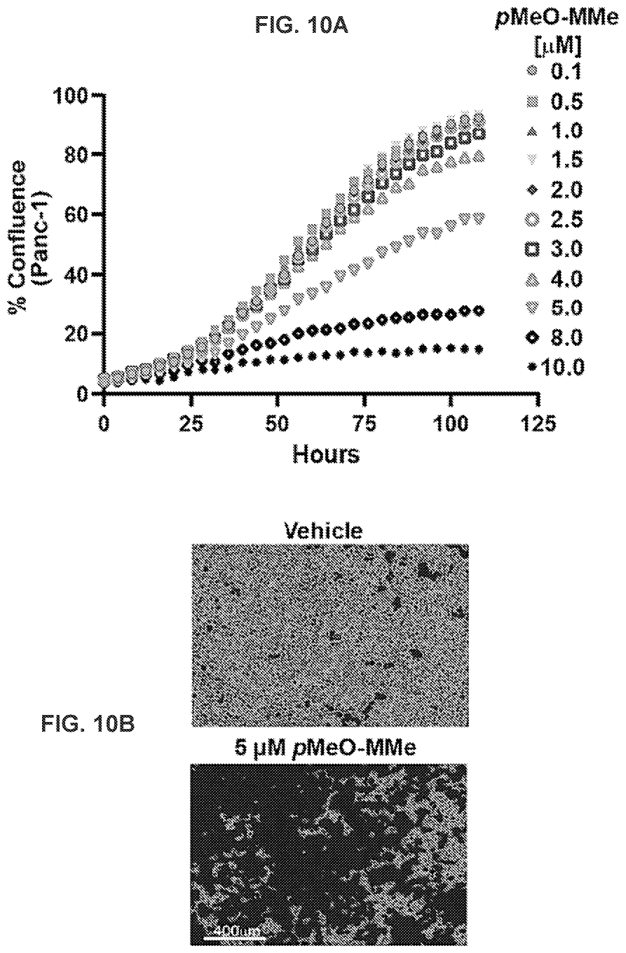

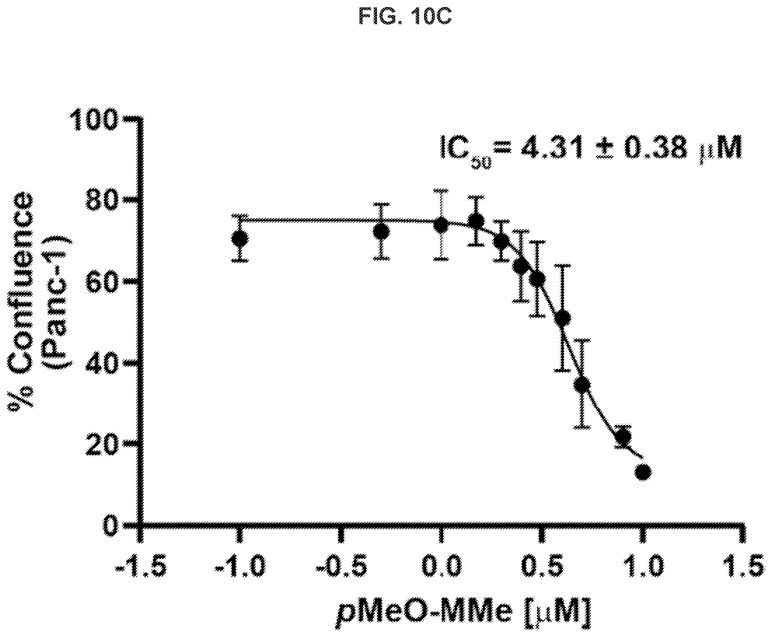

Broad anti-tumorigenic effects of TPP⁺-conjugated compounds were previously reported, with MMe consistently 100-1000-fold more efficacious on pancreatic, breast, and colon cancers compared to the FDA-approved biguanide compound metformin. Here, the anti-tumorigenic effects of the newly synthesized trifluoromethyl and methoxy MMe analogs, pCF₃-MMe, mCF₃-MMe, and pMeO-MMe, were explored on a battery of murine (KPC1242) and human (Panc-1, AsPC-1) pancreatic, colonic (HCT-116), breast (MDA-MB-231), and ovarian (OVCAR-4, OVCAR-8) cancer cell lines. To investigate tumor cell selectivity, human pancreatic nestin-expressing cells (HPNE) were used as a surrogate for normal epithelial cells, and human pancreatic stellate cells (HPSC) were studied as a non-transformed stromal cell type. Cells were exposed to escalating concentrations of MMe, pCF₃-MMe, mCF₃-MMe, and pMeO-MMe (FIGS. 2A-2D) and percent confluency measured as a surrogate for daily proliferation. Growth curves and photomicrographs were used to calculate IC₅₀ values (FIGS. 2A-2D) and confirmed that cell confluency was decreased in a concentration-dependent manner in each of the cancer cells treated with MMe (FIGS. 7A-7H), pCF₃-MMe (FIGS. 8A-8H), and mCF₃-MMe (FIGS. 9A-9J). Examination of pMeO-MMe in human pancreatic cancer cells also revealed a concentration-dependent decrease in confluence (FIGS. 10A-10C).

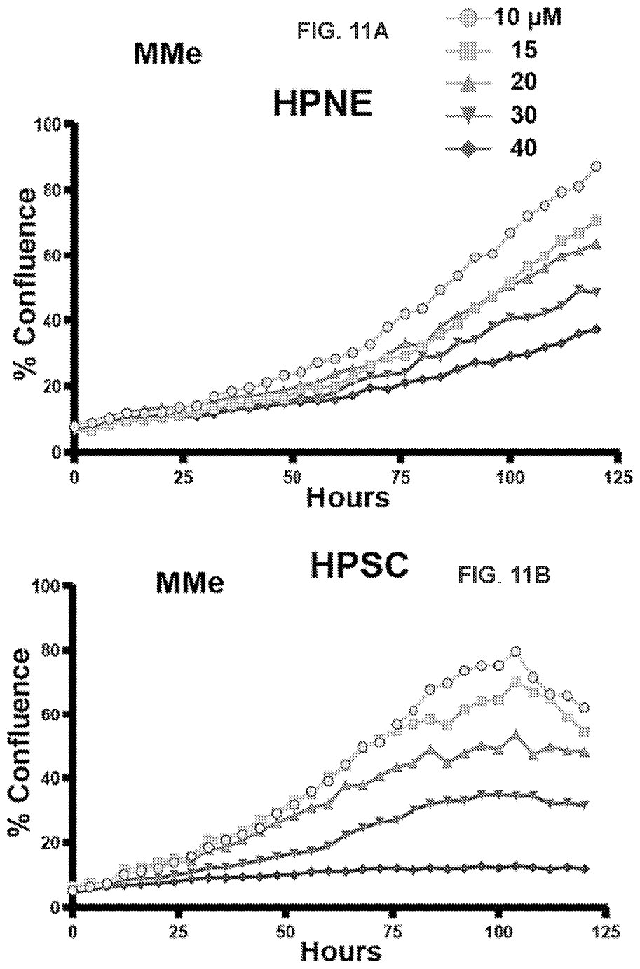

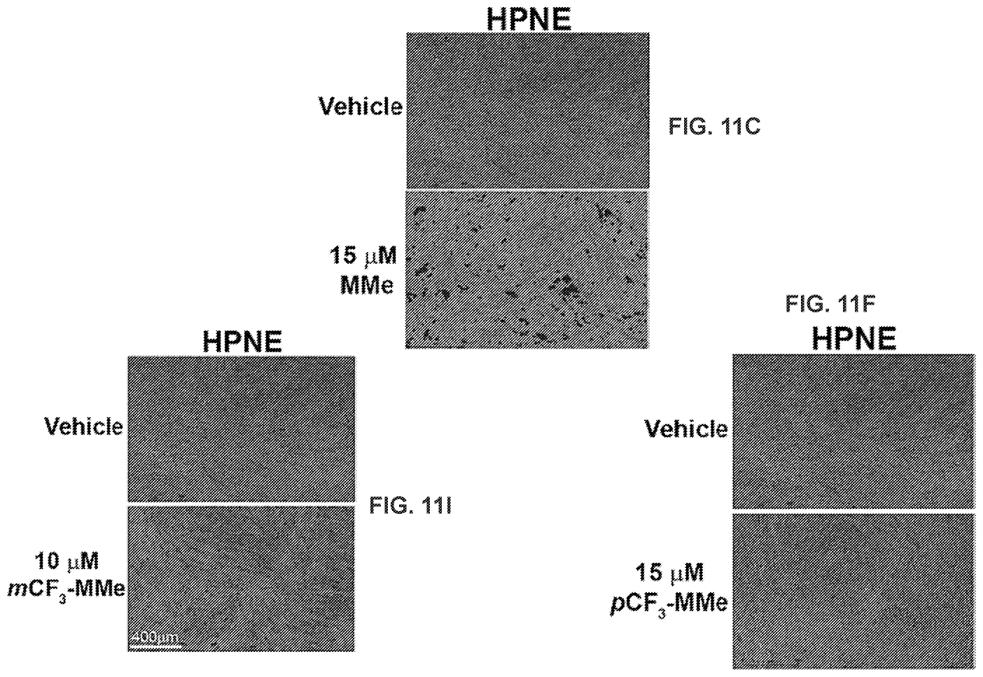

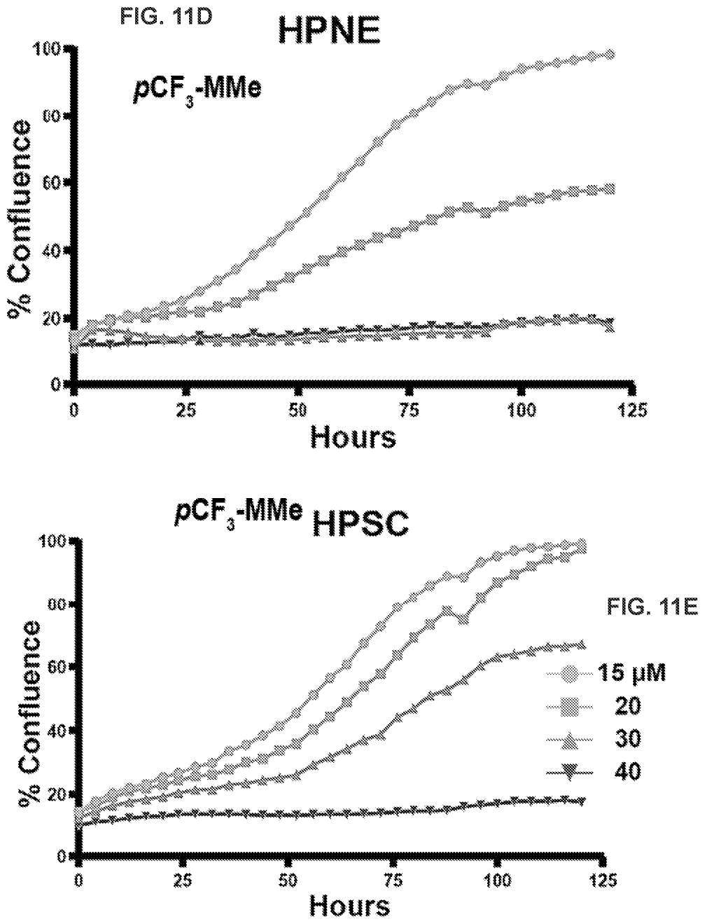

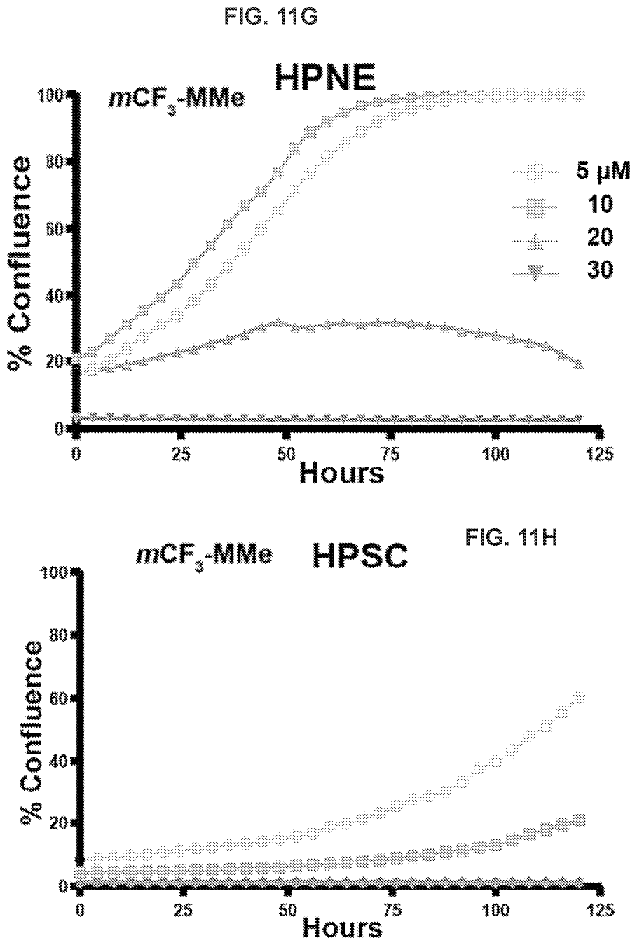

To test the off-target effects of the refined MMe analogs on normal non-transformed cells, the growth of HPNE cells (FIGS. 11A, 11D, and 11G), used as a surrogate for normal pancreatic ductal epithelium, or normal stromal HPSC cells (FIGS. 11B, 11E, and 11H) were measured in increasing concentrations of MMe, pCF₃-MMe, or mCF₃-MMe and compared to the vehicle control. Normal non-transformed cells were more than a log less sensitive to MMe, pCF₃-MMe, and mCF₃-MMe compared to cancer cells (FIGS. 10A-10H). Logarithmic fitting of growth curves revealed strong growth inhibitory effects of MMe (FIGS. 2A-2C), pCF₃-MMe (FIGS. 2D-2F), and mCF₃-MMe (FIGS. 9A and 9B). Moreover, logarithmic fitting of growth curves demonstrated that pCF₃-MMe had the least toxic effects towards non-transformed HPNE and HPSC-1 cells compared to the parent MMe and the mCF₃-MMe analog (FIGS. 2A and 2C; FIG. 9A).

Next, the direct effects of MMe or pCF₃-MMe treatment on cellular proliferation were determined by measuring Ki-67 levels; increased Ki-67 expression is associated with increased cellular growth. KPC1242 cells treated with MMe or pCF₃-MMe demonstrated a significant 20-30% reduction in Ki-67 levels after 24-hour treatment (FIG. 2E). As pCF₃-MMe demonstrated the highest preclinical potential with strong efficacy towards cancer cells and decreased toxicity towards normal cells, we focused on further investigation of pCF₃-MMe.

pCF₃-MMe Maintains Bioenergetic Metabolism Inhibitory Effects of MMe

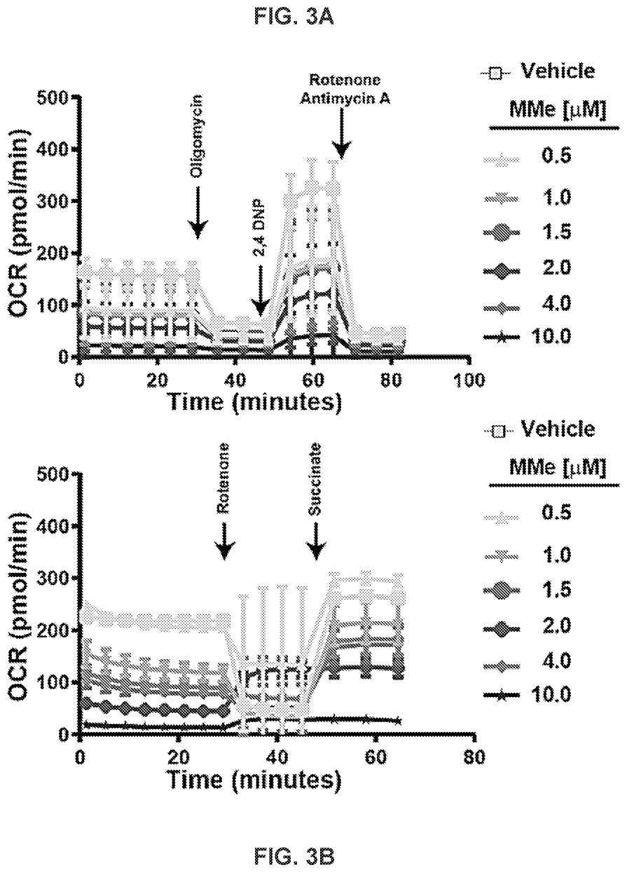

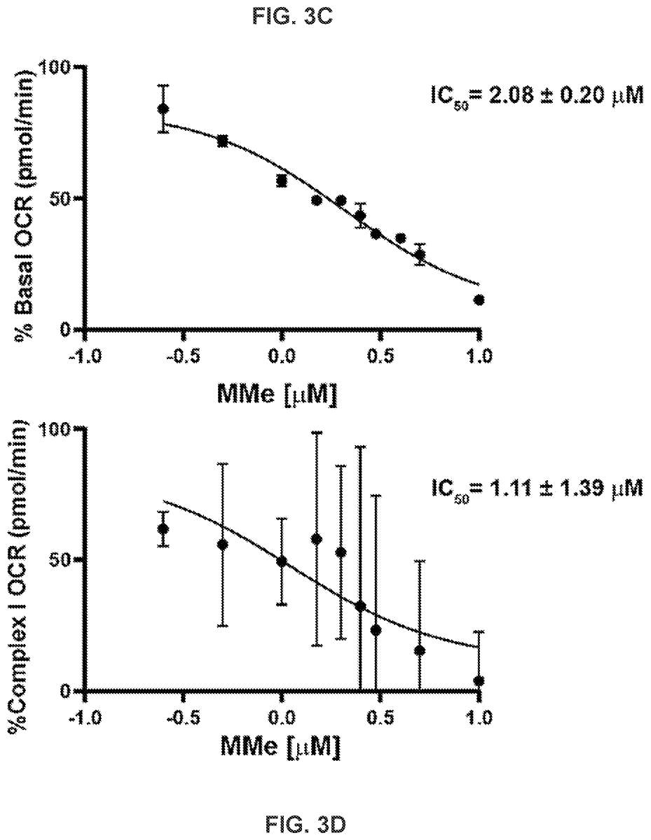

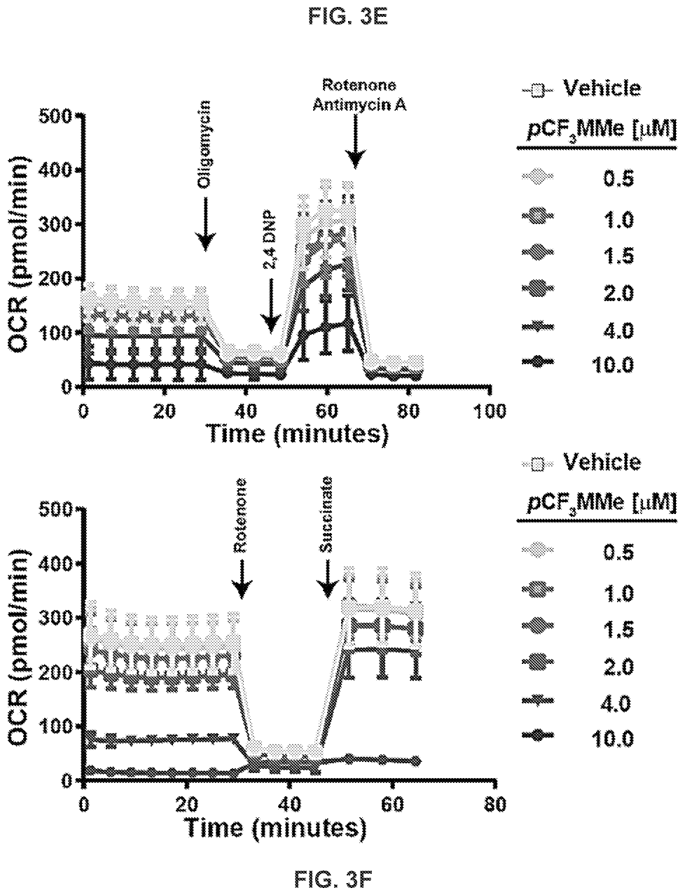

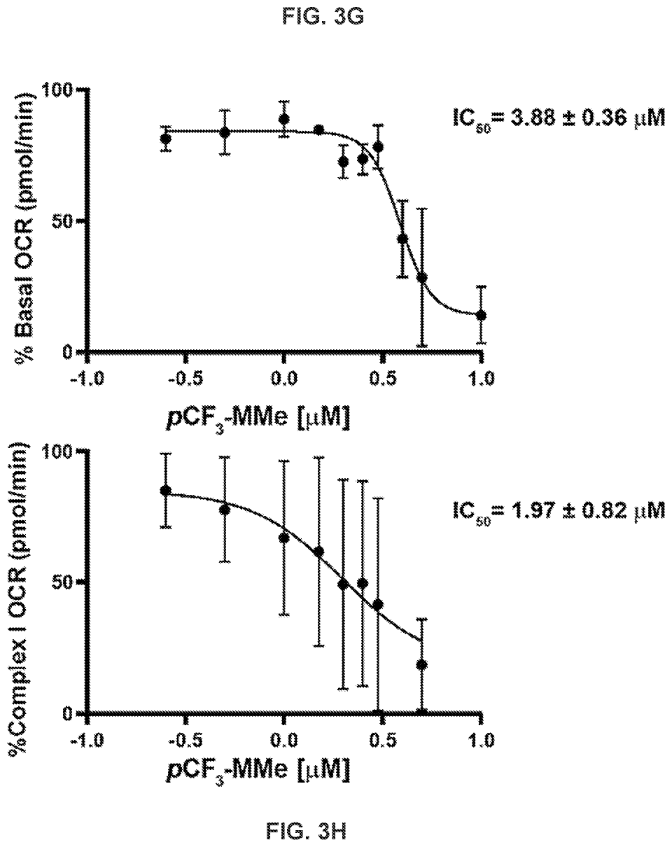

Both the parent compound metformin and mitochondria-targeted compound MMe inhibit complex I mediated oxygen consumption and activate energy-sensing signaling pathways. To determine if the newly synthesized variants retain this functional specificity, further studies were conducted to determine if pCF₃-MMe effectively impacts mitochondrial function and/or induces bioenergetic stress. KPC1242 cells were treated for 24 hours with increasing concentrations of MMe or pCF₃-MMe, and oxygen consumption rate (OCR) was measured as a surrogate for oxidative phosphorylation linked mitochondrial respiration. The Seahorse Cell Mito Stress Test, which provides a quantitative assessment of mitochondrial function that includes basal respiration, ATP-linked respiration, maximal and reserve capacities, and non-mitochondrial respiration, revealed a concentration-dependent decrease in basal OCR, ATP-linked respiration, and maximal respiratory capacity in MMe (FIG. 3A) and pCF₃-MMe (FIG. 3E) treated cells.

Next, further studies were conducted to evaluate if the observed decrease in OCR with pCF₃-MMe corresponded to an inhibition of mitochondrial respiratory complex I. In agreement with previous reports, MMe and pCF₃-MMe decreased complex I derived OCR in the KPC1242 cell line in a concentration-dependent manner. This effect was attenuated by supplementing succinate, a complex II substrate, allowing bypass of complex I, which suggests the mechanism for OCR inhibition is indeed through complex I for both MMe and pCF₃-MMe. Moreover, logarithmic fitting revealed potent basal OCR inhibition (FIGS. 3C and 3G) and complex I inhibition at IC₅₀ values comparable to growth inhibition (FIGS. 3D and 3H).

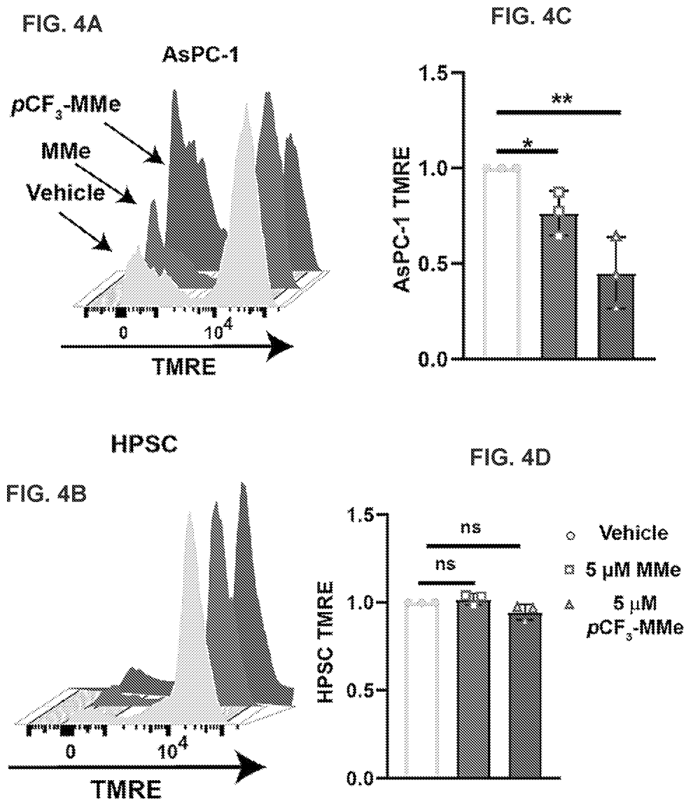

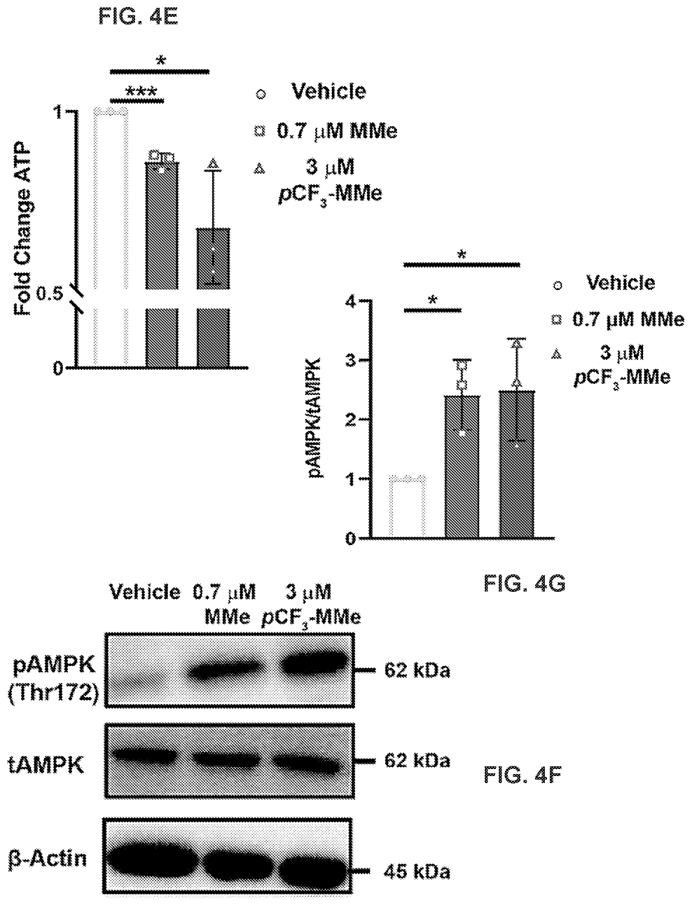

TPP-conjugated compounds are known to effectively target the mitochondria of cancer cells. Thus, the efficacy of pCF₃-MMe to effectively target the mitochondria of cancer cells was tested and compared to normal cells using tetramethylrhodamine ethyl ester perchlorate (TMRE), a cell-permeant, positively-charged dye that accumulates in active mitochondria, and is released into the cytosol upon mitochondrial damage. Differences in TMRE retention are often used to measure mitochondrial damage. As expected and shown in FIGS. 4A-4D, MMe significantly reduced TMRE accumulation within the mitochondria of AsPC-1 measured decreased fluorescence; similarly, pCF₃-MMe significantly reduced TMRE accumulation within the mitochondria of AsPC-1 (FIGS. 4A and 4C). Although not significant, pCF₃-MMe demonstrated a stronger trend towards decreased TMRE accumulation compared to MMe. Consistent with their selectivity towards cancer cells, both MMe and pCF₃-MMe did not affect TMRE accumulation within the HPSC cell line (FIGS. 4B and 4D).

The functional consequence of inhibiting mitochondria complex I and subsequently OXPHOS is a reduction of overall ATP levels in the cell. The ability of MMe to reduce ATP levels was previously reported in numerous cancer cell types. Thus, further studies were conducted to explore if pCF₃-MMe retained similar properties. KPC1242 cells were treated with MMe or pCF₃-MMe for 24 hours at concentrations that inhibit cellular growth and complex I derived OCR (0.7 μM MMe or 3 μM pCF₃-MMe). Levels of ATP were significantly reduced in both MMe and pCF₃-MMe treated cells, consistent with inhibition of metabolic respiration (FIG. 4E). Cells, including cancer cells, must maintain a high ATP:ADP ratio to maintain cellular functions and survival. Moreover, AMP levels rise whenever the ATP:ADP ratio decreases, and an increase in the AMP:ATP ratio signals an energetic crisis in the cell. This energetic crisis is sensed by the master regulator AMP-Activated Protein Kinase (AMPK), which is activated through Thr172 phosphorylation and controls many signaling pathways ranging from energy metabolism to cellular growth. Therefore, further studies were conducted to evaluate if the reduction in ATP levels induced by MMe or pCF₃-MMe is sufficient to activate AMPK. KPC1242 cells treated with 0.7 μM MMe or 3 μM pCF₃-MMe, previously demonstrated to decrease ATP levels (FIG. 4E), showed a significant increase in AMPK activation as quantified by increased Thr172 phosphorylation (FIGS. 4F-4G).

Detection of pCF₃-MMe In Vitro and In Vivo

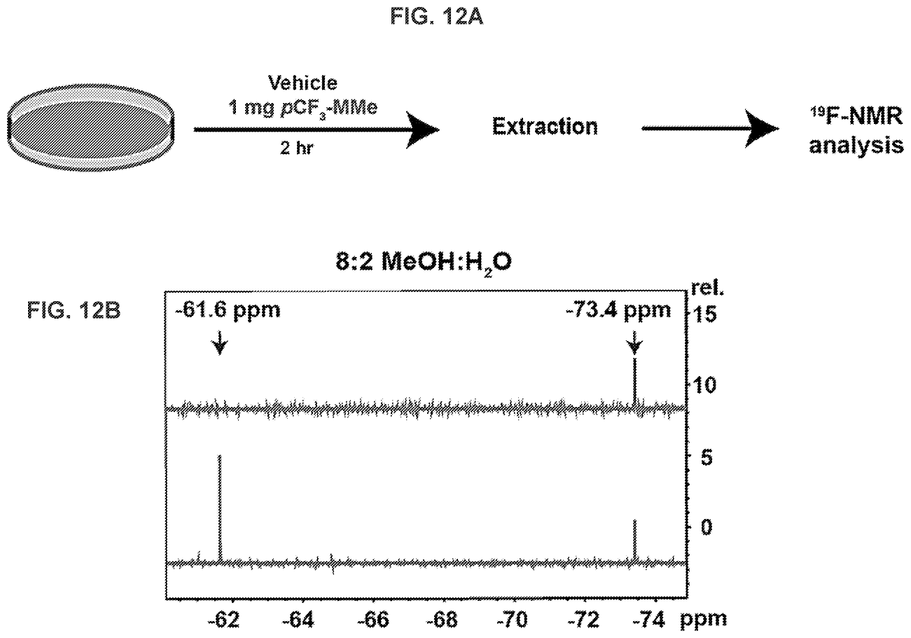

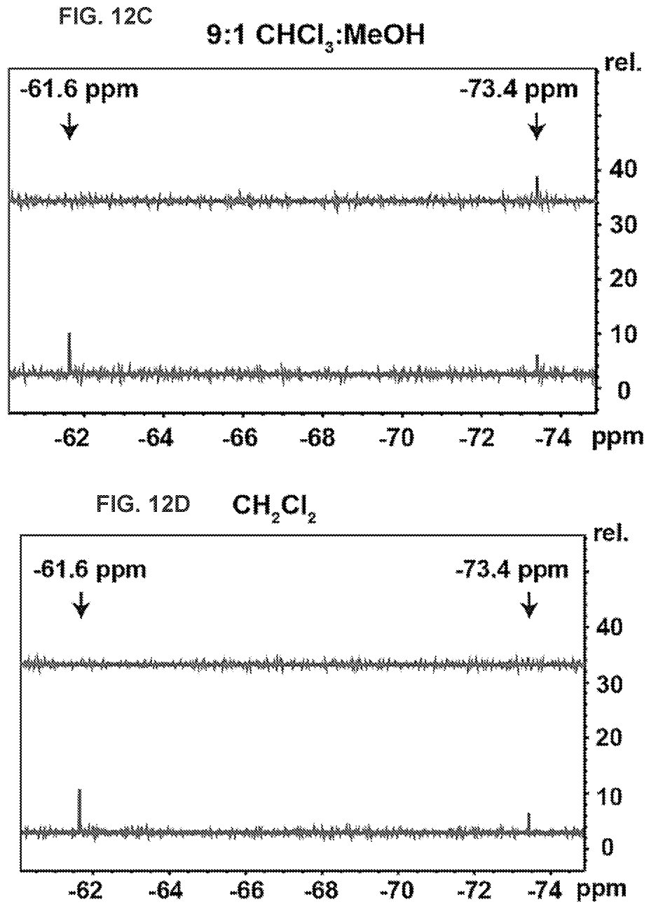

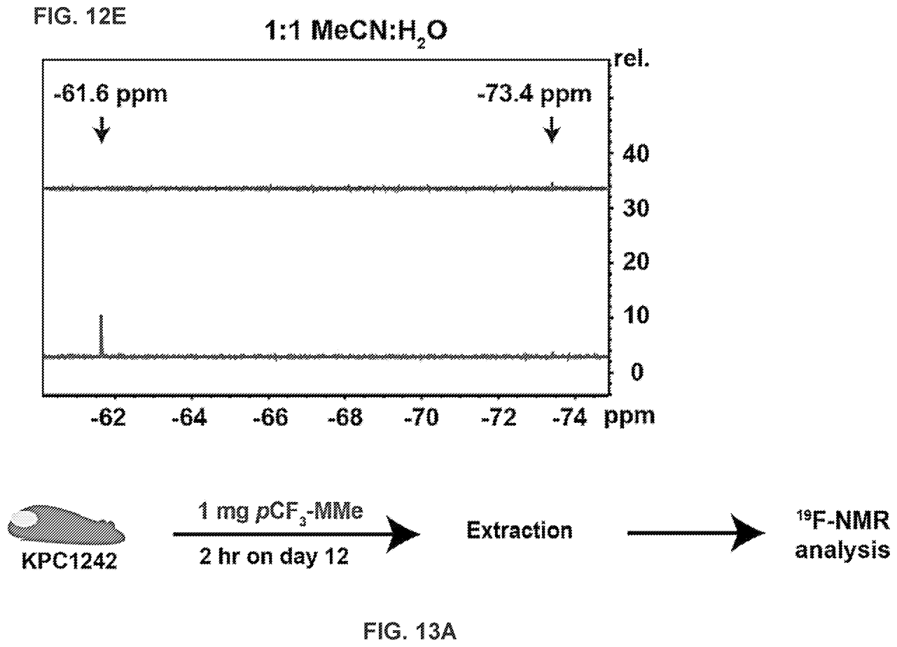

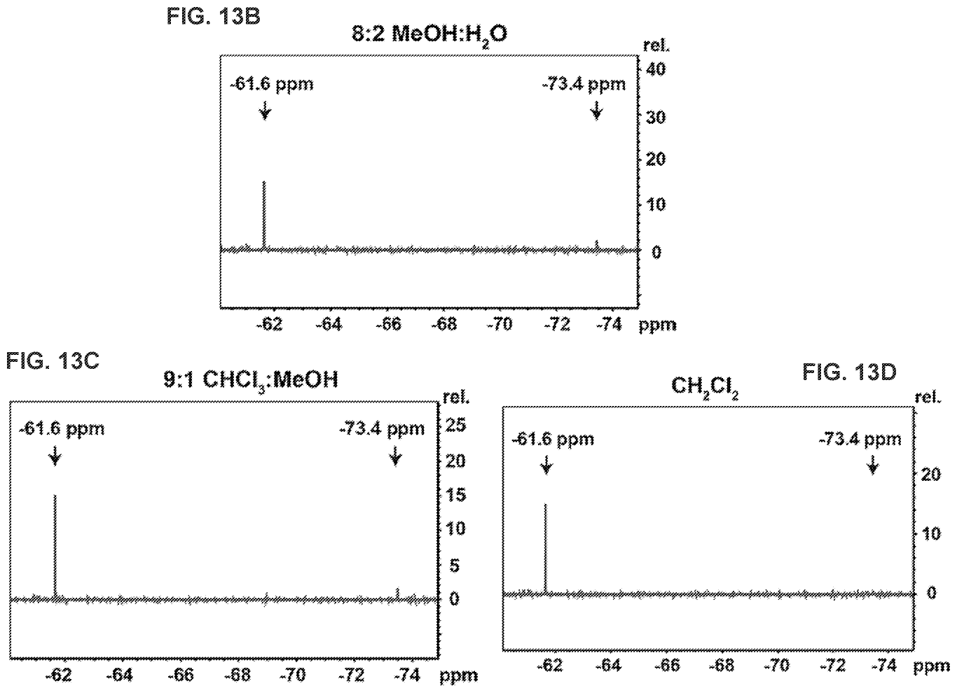

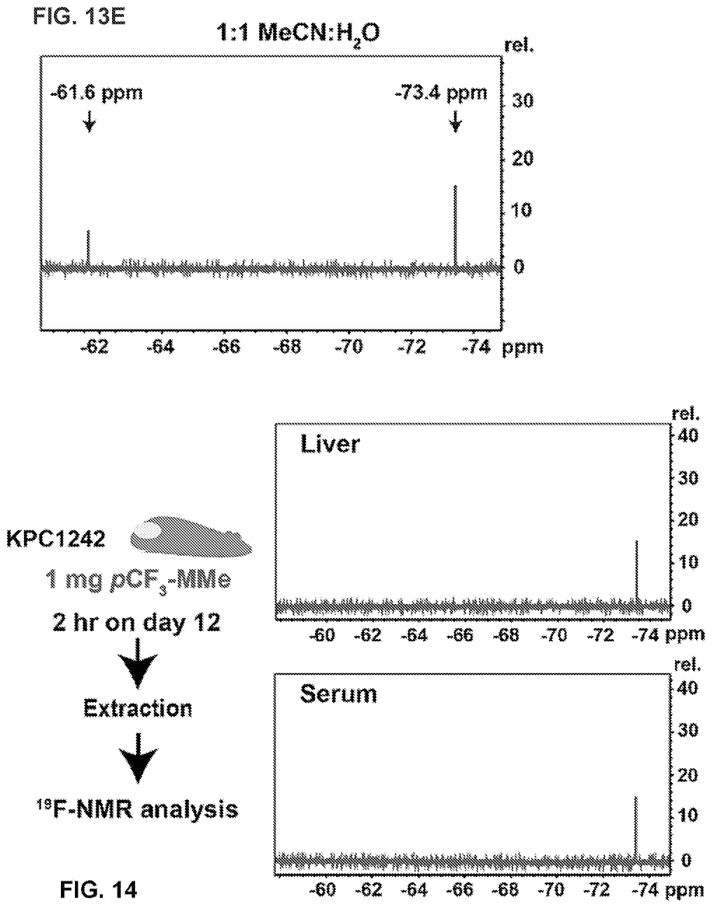

As natural organic compounds containing fluorine are rare in living organisms, trifluoromethyl groups were specifically chosen when designing next-generation MMe analogues. The nine chemically equivalent fluorine atoms in the trifluoromethyl groups of pCF₃-MMe can be studied using ¹⁹F-NMR. To detect cellular accumulation of pCF₃-MMe in vitro, KPC1242 cells were incubated with 1 mg pCF₃-MMe or vehicle (DMSO) in growth media for 2 hours, followed by the removal of the media, compound extraction, and NMR analysis (FIGS. 12A-12E). Four extraction conditions were tested: 8:2 MeOH:H₂O, 9:1 CHCl₃:MeOH, CH₂Cl₂, and 1:1 MeCN:H₂O. MeOH:H₂O (8:2) provided the most efficient extraction, evidenced by the strongest signal observed in ¹⁹F-NMR (−61.6 ppm) among all tested conditions (FIGS. 12B-12E). An additional peak was observed at −73.4 ppm in both the vehicle and pCF₃-MMe treated groups that likely originated from tissue culture plastics as removing the fluorinated plasticizer-containing research consumables completely abolished the signal (FIGS. 12B-12E). To assess in vivo detection of pCF₃-MMe, mice engrafted with KPC1242 cells were administered 1 mg/mouse pCF₃-MMe for 2 hours, followed by tumor harvest and compound extraction (FIG. 13A). NMR analysis demonstrated the consistent detection of pCF₃-MMe, albeit with slightly different efficacy for each of the organic solvents tested (FIGS. 13A-13E). Much like extraction from cell culture, 8:2 MeOH:H₂O extraction was the most efficient for tissues (FIGS. 13B-13E).

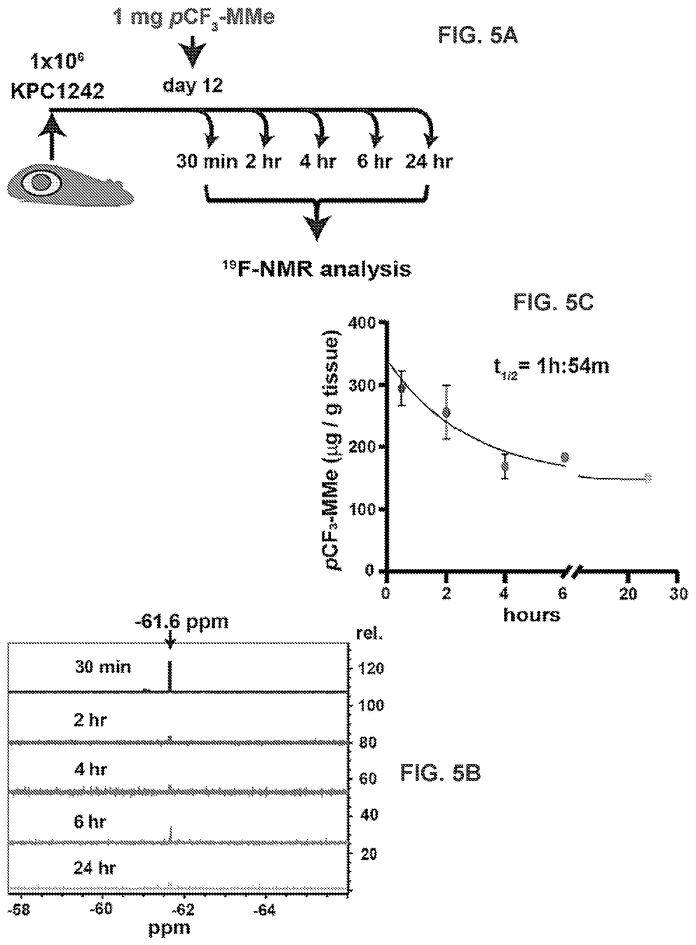

To validate ¹⁹F-NMR as a viable detection approach, the persistence of pCF₃-MMe in tumors was analyzed. KPC1242 engrafted mice were administered 1 mg/mouse pCF₃-MMe starting 12 days after implantation. Mice were sacrificed, and tumors were extracted with 8:2 MeOH:H₂O 30 minutes, 2 hours, 4 hours, 6 hours, or 24 hours after treatment (FIG. 5A). ¹⁹F-NMR revealed measurable levels of pCF₃-MMe within the tumors 24 hours after injection (FIG. 5B). We next designed a quantitative NMR approach to measure the in vivo persistence of pCF₃-MMe. First, serial dilutions of known concentrations of pCF₃-MMe were prepared, and the intensity of the NMR peaks at a set scan number (n=64) was used to create a standard curve. Next, a one-phase decay least-squares fit equation was generated to calculate the amount of pCF₃-MMe at each time point using the standardized scan number. Nonlinear fit curve analysis revealed that pCF₃-MMe had at/2 of 1.9 hours (114 minutes) (FIG. 5C). As an initial study of its pharmacodynamics, quantitative ¹⁹F-NMR indicated that pCF₃-MMe was undetectable in the blood and liver 2 hours after injection (FIG. 14) and was barely detectable above the signal-to-noise ratio at 6 hours (not shown). Taken together, these data reveal that the modification to TPP⁺, particularly by adding chemically equivalent trifluoromethyl groups, allows ease of detection and monitoring of TPP⁺-conjugated compounds.

pCF₃-MMe Attenuates Tumor Progression In Vivo with Superior Tolerability to MMe

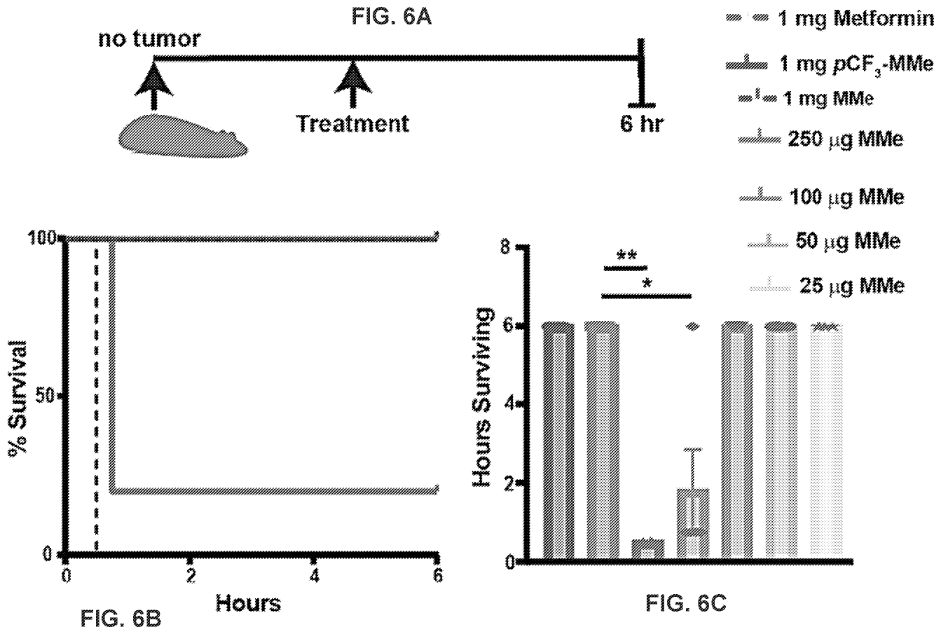

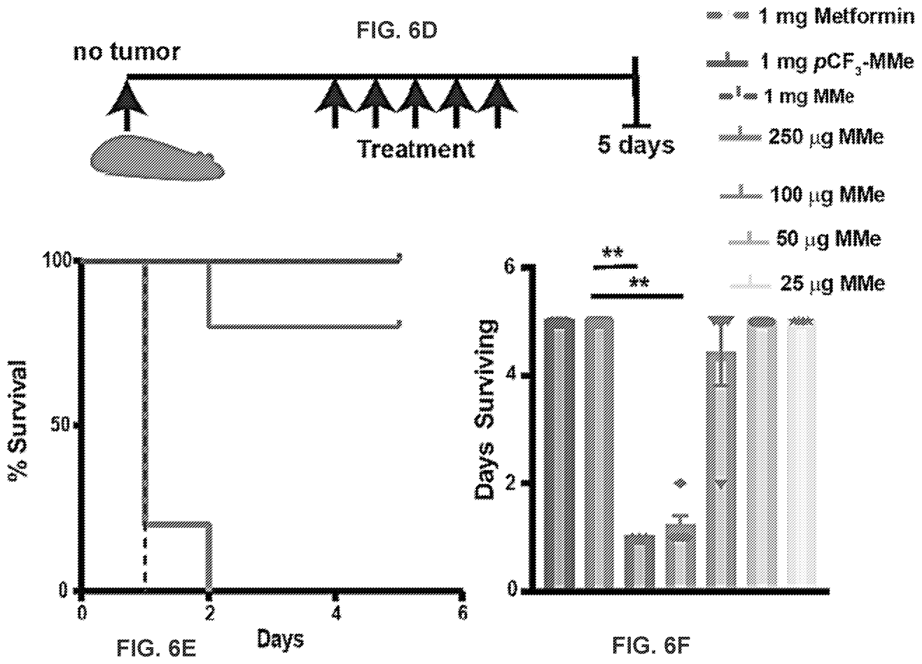

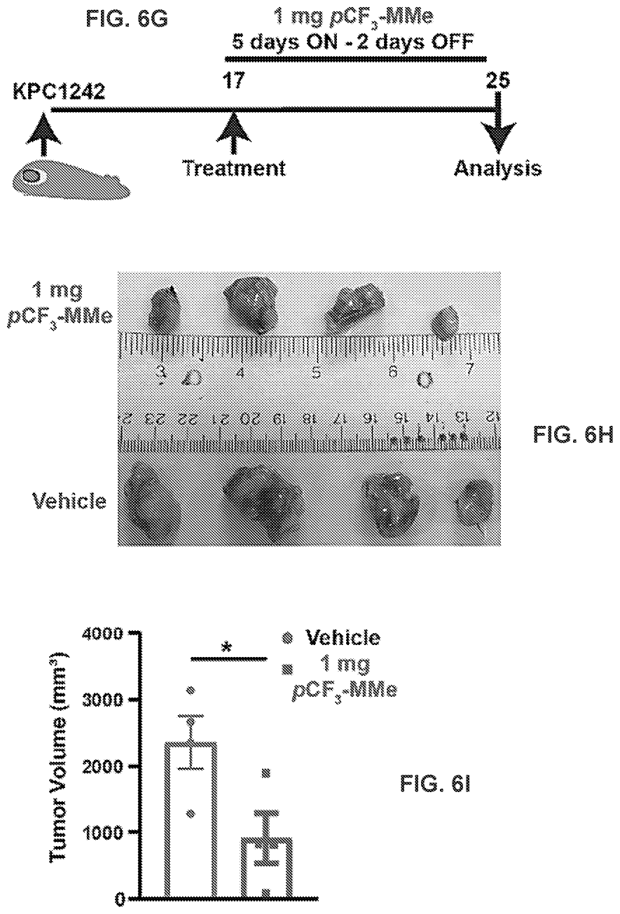

With the ability to detect pCF₃-MMe in vitro and in vivo in hand, along with anti-tumor properties and decreased toxicity towards non-transformed cells in culture, we next compared the tolerability of pCF₃-MMe in mice. Non-tumor-bearing mice received an intraperitoneal injection with decreasing amounts (1 mg, 250 μg, 100 μg, 50 μg, or 25 μg) of unsubstituted MMe or pCF₃-MMe. Additional mice were treated with 1 mg metformin as a treatment control (FIG. 6A). Surprisingly, a single injection of 250 μg or 1 mg MMe to non-tumor-bearing mice resulted in significant and rapid mortality (FIGS. 6B and 6C). In stark contrast, pCF₃-MMe demonstrated vastly superior tolerability, with 100% of mice injected with 1 mg of the fluorinated analog surviving (FIGS. 6B and 6C). Metformin demonstrated a similar safety profile as pCF₃-MMe.

Given the strong safety shown from a single injection, further studies were conducted to determine if mice tolerated repeated injection using our published treatment schedule by injecting non-tumor-bearing mice daily for 5 consecutive days with doses ranging from as little as 25 μg/mouse to as much as 1 mg/mouse 20 (FIG. 6D). Survival curves indicates that while each of the treated mice tolerated a 1 mg dose of pCF₃-MMe, parallel groups succumbed to 4-fold lower doses of un-fluorinated MMe (FIGS. 6E and 6F). Since 1 mg/mouse pCF₃-MMe was well tolerated in tumor-free animals, further studies were conducted to test if pCF₃-MMe could inhibit tumor growth in vivo. Mice were implanted with murine cancer cells and injected 17 days after engraftment with 1 mg pCF₃-MMe (FIG. 6G). Consistent with its anti-proliferative effects in culture, pCF₃-MMe significantly decreased tumor size and volume (FIGS. 6H and 6I). Taken together, trifluoromethylation of TPP⁺ improves the reproducible detection, quantitative measurement, and safety profile of TPP⁺-conjugated compounds while retaining potent anti-tumor effects in vivo.

### Discussion

Cancer cell mitochondrial dysfunction and resulting membrane hyperpolarization have previously been exploited to study and deliver diagnostic and therapeutic agents. While no underlying mechanism has been identified, this property is routinely exploited to selectively detect and target cancer in vitro and in vivo. TPP⁺-conjugated agents take advantage of this hyperpolarization to accumulate in cancer cells and have shown the most promising results in preclinical testing. However, early drug development efforts using TPP⁺ have focused on modifying the aliphatic linker or the cargo. Moreover, the development of TPP⁺-conjugated compounds has been hindered by the lack of sensitive and unbiased approaches to easily detect TPP⁺-conjugated compounds and monitor their distribution in animal models. Herein, TPP⁺-conjugated MMe was redesigned by directly modifying the TPP⁺ moiety to enhance the partial positive charge on the phosphorus atom and increase the lipophilicity of the TPP⁺ moiety. Using cell culture and preclinical models, it was demonstrated that trifluoromethyl-modified MMe has improved selectivity and tolerability with the added benefit of sensitive and quantitative detection of MMe in vivo. These data illustrate that harnessing the electron density of the phosphorus atom in the TPP⁺ provides novel insight into the biological effects of modifying the TPP⁺ moiety.

The large ionic radius of the unsubstituted TPP⁺, mainly due to the delocalized cationic charge on the phosphorus atom, likely plays a role in the ability of TPP⁺ to cross the cellular membrane and target the more negative inside membrane potential of mitochondria. Thus, any substitution on the aryl rings of TPP⁺ will likely impact the ability of these compounds to penetrate the membrane of cancer cells successfully and selectively. A previous report investigated some of the chemical properties of aryl substitution on TPP⁺, lacking the aliphatic carbon chain linker and a cargo, on normal non-transformed C2C12 myoblast cells. However, major knowledge gaps in this report and the field included examining the roles of TPP⁺ aryl substitution on the anti-tumor efficacy when a functional cargo is attached and assessing the preclinical potential of aryl-substituted TPP⁺ compounds. Here, the three TPP⁺ aryl rings were modified to determine if mitochondrial localization and detectability of these reagents could be improved. The three novel compounds described, pCF₃-MMe, mCF₃-MMe, and pMeO-MMe, had distinct antitumor properties. The addition of trifluoromethyl groups in the para position on the TPP⁺ moiety, pCF₃-MMe, was the most optimal modification, with only a slight change in efficacy compared to MMe in several types of cancer. Remarkably, the strongest effect was observed in cell selectivity, with pCF₃-MMe and mCF₃-MMe demonstrating a nearly 10-fold reduction in toxicity to normal cells compared to MMe. These data are consistent with a prior report demonstrating that pCF₃-TPP⁺, a molecule lacking the aliphatic carbon chain linker and bioactive cargo, had no impact on OCR or proliferation of a non-transformed murine myoblast cell line. In contrast to the fluorinated compound, pMeO-MMe demonstrated significantly lower efficacy in pancreas cancer cells. Without being limited to any theory, it is hypothesized that the superior selectivity of pCF₃-MMe towards cancer cells, compared to the electron-donating methoxy groups in pMeO-MMe, reflects the electron-withdrawing groups in the para position, ultimately leading to increased partial positive charge on the phosphorus atom. Further, pCF₃-MMe had the highest octanol/water partition coefficient (c Log P) value indicating increased lipophilicity and, in turn, a potentially improved ability to cross the lipid bilayer and preferentially accumulate in mitochondria membranes. The increase in the partition coefficient of the lipophilic cation may be associated with a greater accumulation of the compound on the matrix side of mitochondria, consistent with the decreased retention of TMRE in pCF₃-MMe, relative to pMeO-MMe, treated cells. While the ratio of the membrane-bound and unbound pools of the compound is defined by the partition constant, the total accumulation at equilibrium is heavily controlled by the membrane potential, as predicted by the Nernst equation, with correction due to membrane/cytosol or matrix partitioning. Thus, changes in the partition coefficient and the electron density may work cooperatively to enhance pCF₃-MMe selectivity toward cancer cells.

The mechanism of action of pCF₃-MMe remained intact relative to the unsubstituted MMe. As shown for the parental compound and several other TPP⁺-conjugated compounds, pCF₃-MMe decreased mitochondrial respiration, inhibited complex I, decreased ATP levels, and activated AMPK signaling in a concentration-dependent manner. These data indicate the para trifluoromethyl substitution did not impact critical binding interactions or decrease the activity of the parent molecule. Importantly, while the para trifluoromethyl aryl substitutions did not interfere with the mechanism of action, this substitution improved the selectivity and tolerability of the molecules in vitro and in vivo. This partly reflects the increase in partial positive charge on the phosphorus atom of pCF₃-MMe, which suggests better accumulation within tumor cells. Previous report showed that using a pCF₃-TPP⁺ substituted on the phenyl rings, in the absence of any conjugated cargo, did not prevent the modified TPP⁺ moiety from accumulating within the mitochondria of intact cells. Indeed, para-substitution of the TPP⁺ phenyl rings led to a higher mitochondrial accumulation of cargo than that mediated by unmodified TPP⁺. Combined, these data demonstrate that the pCF₃-TPP⁺ moiety is a very promising lead for developing mitochondria-targeting agents with increased safety profiles. The results herein support those findings with the added benefit that adding the bioactive cargo metformin to generate pCF₃-MMe retained strong inhibitory effects on mitochondrial respiration. These data suggest that the metformin cargo conjugated to pCF₃-TPP provided the anti-proliferative impact on cancer cells.

Cumulatively, pCF₃-MMe has significant other advantages over traditional unsubstituted TPP⁺-conjugated compounds. First, detection of pCF₃-MMe using ¹⁹F-NMR is greatly enhanced in vitro and in vivo. The ability to quantitative measurement via ¹⁹F-NMR is a marked improvement on the traditional detection of unsubstituted TPP⁺-conjugated compounds requiring laborious a process for detection using LC/MS requiring unique method development for each compound synthesized. Using ¹⁹F-NMR, the half-life of pCF₃-MMe was determined to be −2 hours, a level that closely resembles the pharmacodynamics of many cancer agents. The inability to detect pCF₃-MMe in the blood and liver at 30-minutes or 6 hours supports the idea that the compound is retained at the injection site. A caveat to that interpretation is that metformin is not metabolized and is instead excreted as an intact molecule in the urine. While reports suggest metformin has a half-life of −5 hours, the compound is poorly absorbed, with large inter-individual variability in pharmacokinetics. In contrast to pCF₃-MMe, pharmacokinetic and pharmacodynamic studies of MitoQ, a different, non-fluorinated, TPP⁺-conjugated compound, revealed rapid uptake followed by a sharp decline in the compound after administration, with a half-life of −30 minutes, with the molecule being excreted as an intact molecule in the urine. Yet another advantage is that pCF₃-MMe demonstrated a significantly improved safety profile in vivo similar, with the fluorinated compound demonstrating comparable potency to the FDA-approved parent drug metformin. Additional studies may be conducted to evaluate the pharmacokinetics, pharmacodynamics, and safety profile of TPP⁺-conjugated metformin analogues. The work herein demonstrates that these newly designed compounds may be useful in completing rigorous pharmacology studies. Moreover, pCF₃-MMe opens the door for the non-invasive monitoring of pCF₃-TPP⁺ compounds with complementary techniques such as ¹⁹F-MRI and ¹⁸F-PET.

### Methods

### Electron Density Calculations.

Computational calculations were performed with the Schrodinger Computational Suite (v. 2021-1). Using the LigPrep workflow, all molecules were prepared in the OPLS4 force field at pH=7. Using the Jaguar interface and default settings, electron density and electrostatic potential surfaces of the prepared molecules were calculated using B3LYP-D3 theory and the 6-31G** basis set. Color ramping (minimum and maximum) for the heat map was normalized to unconjugated MMe.

### Cell Culture.

Murine pancreatic cancer cells KPC1242 (David Tuveson, Cold Spring Harbor), human pancreatic cancer cells, Panc-1 (ATCC: CRL-1469), human breast cancer cells MDA-MB-231 (ATCC: CRM-HTB-26), and human pancreatic stellate cells HPSC (Rosa F. Hwang, MD Anderson cancer) were all cultured in DMEM (Gibco ThermoFisher Scientific, Waltham, MA, Cat #11965084) containing 10% v/v fetal bovine serum (Omega Scientific, Inc., Tarzana, CA, Cat #FB-12). Human pancreatic cancer cells AsPC-1 (ATCC: CRL-1682), human ovarian cancer cells OVCAR-8 (NIH: CVCL-1629), and human ovarian cancer cells OVCAR-4 (NIH: CVCL-1627) were all cultured in DMEM (Gibco, Cat #22400089) containing 10% v/v fetal bovine serum (Omega Scientific). Human colon cancer cells HCT-116 (ATCC: CCL-247) were cultured in McCoy's 5A Modified Medium (Gibco, Cat #16600082) containing 10% v/v fetal bovine serum (Omega Scientific). Human pancreatic duct epithelial cells HPNE (ATCC: CRL-4023) were cultured in DMEM and 200 mM L-Glutamine and 7.5% w/v NaCO₃ further supplemented with 15% v/v fetal bovine serum, M3-Base A (Incell, San Antonio, TX), 1M D-Glucose (Gibco Cat #J60067.EQE), 100 μg/mL human EGF (Gibco Cat #PHG0313) and 10 mg/mL puromycin (ThermoFisher, Cat #A1113802).

### Cell Growth and Proliferation.

Cells were plated to standard 96-well tissue culture plates at 4,000 cells/well. Mitochondria-targeted compounds were diluted in full serum medium and added to wells. Cell confluence was measured using an IncuCyte S3 every 2 hours at 10× magnification. To measure proliferation, KPC1242 cells (2×10⁴) were treated with MMe or pCF₃-MMe for 24 hours, fixed with 1% v/v PFA, permeabilized with 0.2% v/v Triton X100, and stained using FITC-conjugated Ki-67 antibody (Cell Signaling Technology, Danvers, MA, Cat #11-5698-82). Image analyses were conducted using the IncuCyte S3 imaging software system.

### Extracellular Flux.

Cells were plated to Seahorse XF96 cell culture microplates at 20,000 cells/well. Stock compounds in DMSO were diluted in RPMI medium to a final concentration in 100 mL volume, incubated at 37° C. for 24 hours, centrifuged at 200×g for 2 minutes, and conditioned medium removed and replaced with serum-free RPMI (200 mL). Oxygen consumption (OCR) and extracellular flux (ECAR) were measured after injections of oligomycin (1 mg/mL), dinitrophenol (50 mM), rotenone (1 mM), and antimycin A (10 mM). Analysis was performed by normalizing OCR and ECAR readouts to vehicle controls. Complex I and II activity were analyzed after permeabilization of the cells.

### ATP Measurements.

KPC1242 cells (2×10⁴) were treated with MMe or pCF₃-MMe for 24 hours. Intracellular ATP levels were determined in cell lysates using a Luminescent ATP Detection Assay Kit per the manufacturer's directions (Abcam, Cambridge, UK, Cat #ab113849).

### Immunoblotting.

Cells were serum-starved for 24 hours followed by compound treatment for 24 hours before lysis in RIPA buffer composed of 50 mM Tris-HCl, pH 7.4, 150 mM NaCl, 0.25% v/v sodium deoxycholate, 1% v/v Nonidet P-40, 0.1% v/v SDS, 1 mM EDTA, 10 mM sodium orthovanadate, 40 mM 0-glycerol phosphate, 20 mM sodium fluoride, and protease inhibitors cocktail set III (Millipore Sigma, Cat #539134). Protein (10 μg) was loaded to each lane and separated by reducing SDS-PAGE. The separated proteins were electro-transferred to PVDF membranes and incubated with primary anti-phospho-AMPKα or anti-total AMPKα (Cell Signaling Technology #2531S or #2532S, respectively) or anti-R-actin (Cell Signaling, Cat #4970S). Primary antibodies were detected by incubating with secondary horseradish peroxidase-conjugated antibody (Cytiva, Amersham ECL rabbit IgG-HRP, Cat #NA9340). Densitometric quantification of phosphorylated, total or actin protein levels was measured using chemiluminescence and calculated using Alpha View Software.

### Sample Preparation and NMR Analysis.

Cells (1×10⁷ cells/10 cm dish) were allowed to adhere overnight and then treated with varying concentrations of mitochondria-targeted compounds. Compounds were extracted using multiple methods and optimized for detection and quantification: A) Cells or tumors were placed in a dry ice ethanol bath followed by 80% v/v MeOH in water. Cells were placed on a dry ice ethanol bath on an orbital shaker for 20 minutes, and then cells were collected and centrifuged at 10,000×g; B) Post-treatment cells were lifted with TrypLE (Gibco, Cat #11558856) and centrifuged, then compound was extracted from the cells or tumors with 9:1 CHCl₃:H₂O, CH₂Cl₂, or 1:1 MeCN:H₂O. Following extraction, solvents were evaporated using a Biotage V-10 Touch (Uppsala, Sweden), and before NMR acquisition, samples were resuspended in an equal volume of DMSO-d6.

### NMR Data Collection.

¹⁹F 1D-NMR spectra were collected on a Bruker Avance-III 500 MHz NMR spectrometer equipped with a 1H&¹⁹F/13C/15N-TCI cryoprobe. All ¹⁹F data were collected at 25° C. using a 1D pulse sequence with a 30° flip-angle, 64 transients, and an automatic receiver gain. TopSpin 3.6.1 was used to process all spectra and integrate the peak intensity corresponding to the pCF₃-MMe ¹⁹F signal at −61.6 ppm. To evaluate pCF₃-MMe concentrations in biological samples, a standard curve was generated using pCF₃-MMe concentrations of 0.25, 0.50, 0.75, 1.0, 1.25, and 1.5 mg/mL, and the resulting peak intensities were analyzed by linear regression. The standard curve was then used to calculate the amount of pCF₃-MMe present per gram of tissue in the processed biological samples.

### Animal Models.

All animal studies were approved by the Institutional Animal Care and Use Committees at the Medical College of Wisconsin. C57BL/6 mice (RRID: IMSR JAX:000664) were purchased from The Jackson Laboratory (Bar Harbor, ME). Six- to eight-week-old male or female mice were randomly assigned to treatment or control groups and implanted subcutaneously in the right flank with 1×10⁶ KPC1242 murine pancreatic cancer cells. Tumors were allowed to establish for 12-17 days before intra-tumoral treatment. Mice were randomly assigned to control or experimental treatment groups immediately before treatment by an investigator blinded to the ex vivo tumor measurements. Tumors were excised, and volume (length×width×depth=mm³) was measured post-mortem using calipers. Tumors were then fixed in zinc formalin and cleared in 70% v/v ethanol, embedded in paraffin, and 7 μm sections stained for immunohistochemical analysis.

### Statistical Analysis.

All statistical analyses were performed using GraphPad Prism (La Jolla, CA). Paired analyses were calculated using Student's t-test to identify pair-wise differences between experimental and control groups. Values provided represent mean±S.D. Statistical significance was defined as p≤0.05 and denoted with asterisks as indicated in the figure legends.

### Example 2. Supplemental Information for Example 1

### General Compound Synthesis Methods and Equipment.

All reagents and solvents were commercial grade and purified before use. Thin layer chromatography was performed using glass-backed silica gel (250 μm) plates. UV light and/or iodine, potassium permanganate, and potassium iodoplatinate stains were used to visualize products. MPLC was performed on a Biotage Isolera in conjunction with a Biotage Dalton 2000 using the conditions indicated. Nuclear magnetic resonance spectra (NMR) were acquired on a Bruker Avance-III-500 MHz spectrometer equipped with a TCI cryoprobe. 1H and 13C chemical shifts were measured relative to residual solvent peaks as an internal standard set to δ 7.26 and δ 77.0 (CDCl3), δ 2.50 and δ 39.5 (DMSO-d6), or δ 3.31 and δ 49.00 (CD3OD). Low-resolution mass spectra were recorded on a Biotage Dalton 2000 or Advion Expression Compact Mass Spectrometer using the indicated ionization method. High-resolution mass spectra were recorded at the Indiana University Mass Spectrometry Facility on a Thermo Scientific Orbitrap XL spectrometer using the indicated ionization method. Post-acquisition gain correction was applied using reserpine as a lock mass.

### General Procedure-A for the Preparation of Compounds 2a-d

N-(10-bromodecyl)phthalimide (1 equivalent) was dissolved in anhydrous CH₃CN or DMF (0.32 M) and placed under nitrogen. This was treated with the indicated phosphine (1 equivalent). The reaction was then heated at reflux for CH₃CN or in a pressure vessel at 140° C. for DMF for 18-51 hours. The reaction was then concentrated in vacuo to give crude 2a-d. The material was purified by silica column chromatography (0-30% v/v MeOH/DCM) to provide the title compound.

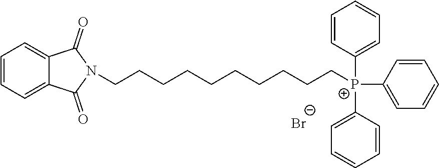 [SMILES: O=C1c2ccccc2C(=O)N1CCCCCCCCCC[P+](c1ccccc1)(c1ccccc1)c1ccccc1 ; [Br-]]

[10-(1,3-dioxo-2,3-dihydro-1H-isoindol-2-yl)decyl]triphenylphosphonium bromide (2a). A mixture of 2.5 g (6.83 mmol) of N-(10-bromodecyl)phthalimide in CH3CN (21 mL) was refluxed with triphenylphosphine for 18 hours. Flash chromatography gave 3.98 g (93%) of the title compound. Spectra match those previously reported¹.

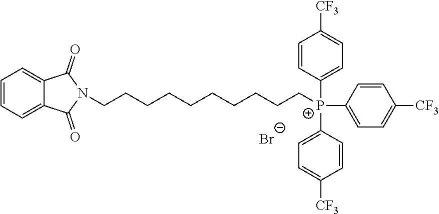 [SMILES: O=C1c2ccccc2C(=O)N1CCCCCCCCCC[P+](c1ccc(C(F)(F)F)cc1)(c1ccc(C(F)(F)F)cc1)c1ccc(C(F)(F)F)cc1 ; [Br-]]

[10-(1,3-dioxo-2,3-dihydro-1H-isoindol-2-yl)decyl]tris[4-(trifluoromethyl)phenyl]-phosphonium bromide (2b). A mixture of 1.5 g (4.10 mmol) of N-(10-bromodecyl)phthalimide in DMF (12.6 mL) was heated with tris(4-trifluoromethyl)phosphine (1.91 g, 4.10 mmol) for 51 hours. Flash chromatography gave 2.59 g (76%) of the title compound. Spectra match those previously reported¹.

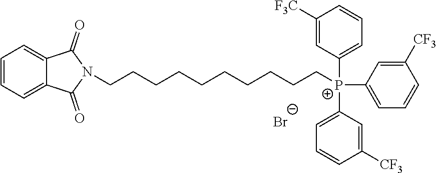 [SMILES: O=C1c2ccccc2C(=O)N1CCCCCCCCCC[P+](c1cccc(C(F)(F)F)c1)(c1cccc(C(F)(F)F)c1)c1cccc(C(F)(F)F)c1 ; [Br-]]

[10-(1,3-dioxo-2,3-dihydro-1H-isoindol-2-yl)decyl]tris[3-(trifluoromethyl)phenyl]-phosphonium bromide (2c). A mixture of 0.500 g (1.37 mmol) of N-(10-bromodecyl)phthalimide in DMF (12.6 mL) was heated with tris(3-trifluoromethyl)phosphine 2 (0.637 g, 1.37 mmol) for 51 hours. Flash chromatography gave 0.442 g (76%) of the title compound.

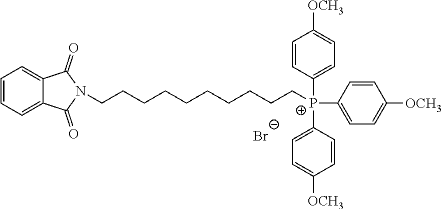 [SMILES: COc1ccc([P+](CCCCCCCCCCN2C(=O)c3ccccc3C2=O)(c2ccc(OC)cc2)c2ccc(OC)cc2)cc1 ; [Br-]]

[10-(1,3-dioxo-2,3-dihydro-1H-isoindol-2-yl)decyl]tris(4-methoxyphenyl)phosphonium bromide (2d). A mixture of 0.500 g (1.37 mmol) of N-(10-bromodecyl)phthalimide in CH₃CN (4.2 mL) was refluxed with triphenylphosphine for 19 hours. Flash chromatography gave 0.920 g (93%) of the title compound. 1H NMR (500 MHz, DMSO) δ 7.92-7.85 (m, 4H), 7.69 (dd, J=10.3, 10.3 Hz, 6H), 7.32 (dd, J=2.1, 8.8 Hz, 6H), 3.92 (s, 9H), 3.58 (t, J=7.1 Hz, 2H), 1.59 (m, J=7.1 Hz, 2H), 1.54-1.40 (m, 5H), 1.32-1.17 (m, 11H).

### General Procedure-B for the Preparation of Compounds 4a-c

Compound 2a-c (1 equivalent) was dissolved in EtOH (0.16 M) and placed under nitrogen. This was treated with hydrazine (1 equivalent or 1.5 equivalent). The reaction was then heated to reflux for 20 hours. The reaction was then concentrated, and the byproduct was precipitated using EtOH/Et2O and removed by vacuum filtration to give crude intermediate 3a-d. The material was purified by silica column chromatography using amine-treated silica (0-20% v/v MeOH/DCM). The residue (1 equivalent) was dissolved in n-BuOH (0.24 M) and placed under nitrogen. This was treated with N-[amino(methylsulfanyl)methylidene)guanidine hydroiodide 3 (1.15 equivalent). The reaction was heated to 130° C. for 3 hours and concentrated. The material was purified by silica column chromatography using amine-treated silica (1-20% v/v MeOH/DCM) to give the title compound.

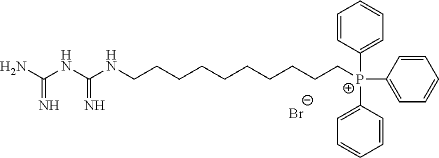 [SMILES: N=C(N)NC(=N)NCCCCCCCCCC[P+](c1ccccc1)(c1ccccc1)c1ccccc1 ; [Br-]]

10-[(carbamimidamidomethanimidoyl)amino]decyl-triphenylphosphonium bromide (4a, MMe). A mixture of 2a (1.5 g, 2.39 mmol) and hydrazine (0.074 mL, 2.39 mmol) was refluxed in ethanol (15 mL) for 20 hours. Purification by silica column chromatography afforded the amine as an amorphous solid, which was carried forward without further manipulation. LRMS (+ESI) m/z [M−Br]+, Calculated for C28H37NP 418.3; Found 418.4. A mixture of 0.906 g (1.82 mmol) of (10-aminodecyl)triphenylphosphonium in n-BuOH (7.5 mL) was heated with N-[amino(methylsulfanyl)methylidene)guanidine hydroiodide (0.543 g, 2.09 mmol). Flash chromatography gave 0.367 g (35%) of the title compound as an amorphous solid. HRMS (+ESI) m/z: [M−Br]+ calculated for C30H41N5P 502.3094; Found 502.3094.

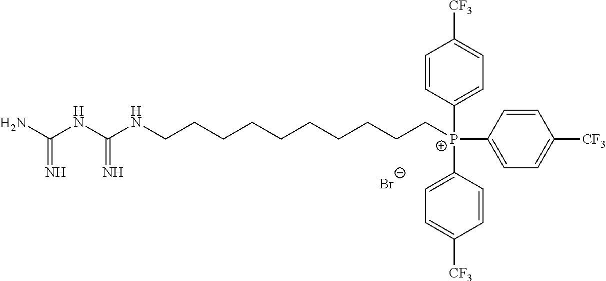 [SMILES: N=C(N)NC(=N)NCCCCCCCCCC[P+](c1ccc(C(F)(F)F)cc1)(c1ccc(C(F)(F)F)cc1)c1ccc(C(F)(F)F)cc1 ; [Br-]]

(10-(3-carbamimidoylguanidino)decyl)tris(4-(trifluoromethyl)phenyl)phosphonium bromide (4b, pCF₃-MMe). A mixture of 2b (2.59 g, 3.11 mmol) and hydrazine (0.145 mL, 4.66 mmol) was refluxed in ethanol (19 mL) for 20 hours. Purification by silica column chromatography afforded the amine as an amorphous solid, which was carried forward without further manipulation. LRMS (+ESI) m/z [M−Br]+, Calculated for C31H34F9NP 622.2; Found 622.2. A mixture of 0.630 g (0.879 mmol) of (10-aminodecyl)tris[4-(trifluoromethyl)phenyl]phosphonium bromide in n-BuOH (3.7 mL) was heated with N-[amino(methylsulfanyl)methylidene)guanidine hydroiodide (0.268 g, 1.03 mmol). Flash chromatography gave 0.224 g (32%) of the title compound as an amorphous solid. 1H NMR (500 MHz, CD3OD) δ 8.06-7.73 (12H, m), 3.26-3.19 (2H, m), 3.12-3.05 (1H, m), 2.97-2.90 (4H, m), 2.51-2.40 (1H, m), 1.55-1.29 (6H, m), 1.29-1.10 (10H, m). ¹⁹F NMR (470 MHz, CD3OD) δ −64.66. HRMS (+ESI) m/z: [M−Br]+ calculated for C33H38F9N5P 706.2716; Found 706.2722.

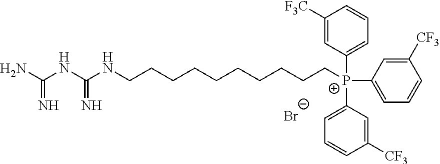 [SMILES: N=C(N)NC(=N)NCCCCCCCCCC[P+](c1cccc(C(F)(F)F)c1)(c1cccc(C(F)(F)F)c1)c1cccc(C(F)(F)F)c1 ; [Br-]]

(10-(3-carbamimidoylguanidino)decyl)tris(3-(trifluoromethyl)phenyl)phosphonium bromide (4c, mCF₃-MMe). A mixture of 2c (0.4421 g, 0.531 mmol) and hydrazine (0.025 mL, 0.796 mmol) was refluxed in ethanol for 20 hours. Purification by silica column chromatography afforded the amine as an amorphous solid, which was carried forward without further manipulation. LRMS (+ESI) m/z [M−Br]+, calculated for C31H34F9NP 622.2; Found 622.2. A mixture of 0.069 g (0.098 mmol) of (10-aminodecyl)tris[3-(trifluoromethyl)phenyl]phosphonium bromide in n-BuOH (1 mL) was heated with N-[amino(methylsulfanyl)methylidene)guanidine hydroiodide (0.029 g, 0.113 mmol). Flash chromatography gave 0.006 g (7%) of the title compound as an amorphous solid. 1H NMR (500 MHz, DMSO-d6) δ 8.40-7.62 (6H, m), 6.71-6.53 (10H, m), 1.64-1.14 (16H, m). ¹⁹F NMR (470 MHz, CD3OD) δ −61.19. Significant 31P splitting complicated the proton spectrum. HRMS (+ESI) m/z: [M−Br]+ Calculated for C33H38F9N5P 706.2716; Found 706.2713.

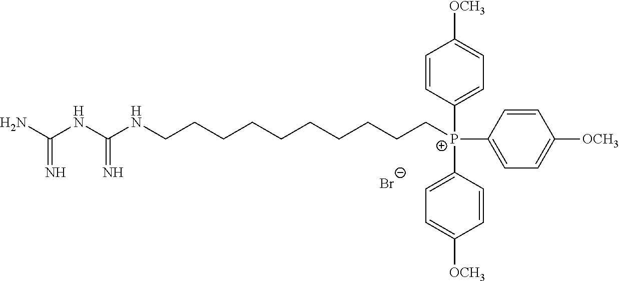 [SMILES: COc1ccc([P+](CCCCCCCCCCNC(=N)NC(=N)N)(c2ccc(OC)cc2)c2ccc(OC)cc2)cc1 ; [Br-]]

[10-(2-carbamimidoylethanimidamido)decyl]tris(4-methoxyphenyl)phosphonium bromide (4d, pMeO-MMe). Compound 2d (0.920 g, 1.28 mmol) was dissolved in EtOH (8 mL) and placed under nitrogen. This was treated with hydrazine (0.040 mL, 1.28 mmol). The reaction was then heated to reflux for 20 hours. The reaction was then concentrated, and the byproduct was precipitated using EtOH/Et2O and removed by vacuum filtration. The filtrate was concentrated in vacuo to give crude intermediate 3d. Intermediate 3d (0.150 g, 0.255 mmol) was dissolved in CH₂Cl₂ (2.5 mL) and cooled to 0° C. in an ice bath. This was treated with hydrogen chloride (0.765 mL, 0.765 mmol, 1 M in Et20) by dropwise addition. Once the addition was complete, the reaction was stirred at room temperature for 1 hour. The reaction was then concentrated in vacuo. The resulting yellowish foam was dissolved in anhydrous DMF (1.0 mL) and treated with diacyldiamide (0.096 g, 1.47 mmol). The reaction was then heated to 160° C. for 2 hours. The reaction was concentrated in vacuo. The material was purified by silica column chromatography using amine-treated silica (1-30% v/v MeOH/DCM) to give 0.114 g (67%) of the title compound. 1H NMR (500 MHz, CD3OD) δ 7.61-7.47 (6H, m), 7.21-7.08 (6H, m), 3.83 (9H, s), 3.26-3.19 (2H, m), 3.12-3.05 (3H, m), 3.02-2.91 (6H, m), 1.60-1.50 (2H, m), 1.47-1.35 (4H, m), 1.30-1.11 (10H, m). HRMS (+ESI) m/z: [M−Br]+ calculated for C33H47N3O5 592.3411; Found 592.3409.

### Schematic Synthesis Pathway for Generating TPP⁺-Conjugated MMe Analogues.

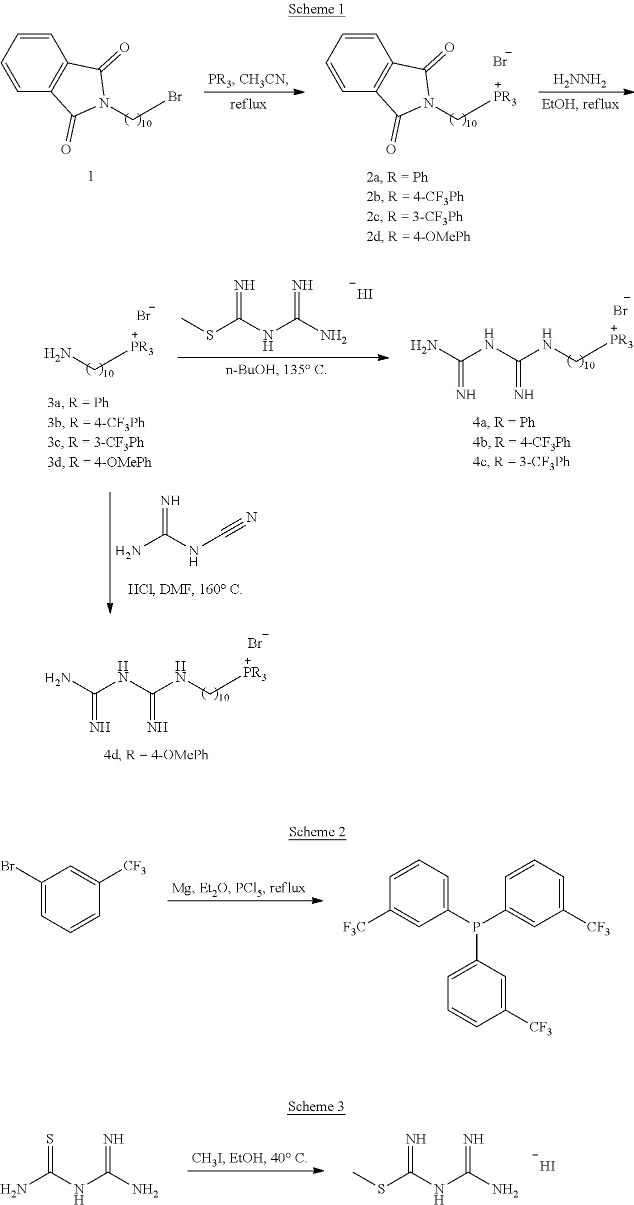

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

### REFERENCES

1. Smith, R. A., Porteous, C. M., Gane, A. M. & Murphy, M. P. Delivery of bioactive molecules to mitochondria in vivo. Proc Natl Acad Sci USA. 100, 5407-5412. doi: 5410.1073/pnas.0931245100. Epub 09312423 April 0931245115. (2003).
2. Frattaruolo, L. et al. Targeting the Mitochondrial Metabolic Network: A Promising Strategy in Cancer Treatment. Int J Mol Sci. 21, 6014. doi: 6010.3390/ijms21176014. (2020).
3. Rovini, A. et al. Quantitative analysis of mitochondrial membrane potential heterogeneity in unsynchronized and synchronized cancer cells. FASEB J. 35, e21148. doi: 21110.21096/fj.202001693R. Epub 20200220 November 202001616. (2021).
4. Momcilovic, M. et al. in vivo imaging of mitochondrial membrane potential in non-small-cell lung cancer. Nature. 575, 380-384. doi: 310.1038/s41586-41019-41715-41580. Epub 4219 October 41530. (2019).
5. Armstrong, J. S. Mitochondrial medicine: pharmacological targeting of mitochondria in disease. Br J Pharmacol. 151, 1154-1165. doi: 1110.1038/sj.bjp.0707288. Epub 0702007 May 0707221. (2007).
6. Zong, W. X., Rabinowitz, J. D. & White, E. Mitochondria and Cancer. Mol Cell. 61, 667-676. doi: 610.1016/j.molcel.2016.1002.1011. (2016).
7. Pustylnikov, S., Costabile, F., Beghi, S. & Facciabene, A. Targeting mitochondria in cancer: current concepts and immunotherapy approaches. Transl Res. 202:35-51., 10.1016/j.trsl.2018.1007.1013. Epub 2018 July 1031. (2018).
8. Cheng, G. et al. Mitochondria-targeted drugs synergize with 2-deoxyglucose to trigger breast cancer cell death. Cancer Res 72, 2634-2644 (2012). https://doi.org:10.1158/0008-5472.CAN-11-3928
9. Cheng, G. et al. Mitochondria-Targeted Analogues of Metformin Exhibit Enhanced Antiproliferative and Radiosensitizing Effects in Pancreatic Cancer Cells. Cancer Res 76, 3904-3915 (2016). https://doi.org:10.1158/0008-5472.CAN-15-2534.
10. Cheng, G. et al. Synergistic inhibition of tumor cell proliferation by metformin and mito-metformin in the presence of iron chelators. Oncotarget 10, 3518-3532 (2019). https://doi.org:10.18632/oncotarget.26943.
11. Boyle, K. A. et al. Mitochondria-targeted drugs stimulate mitophagy and abrogate colon cancer cell proliferation. The Journal of biological chemistry 293, 14891-14904 (2018). https://doi.org:10.1074/jbc.RA117.001469.
12. Zielonka, J. et al. Mitochondria-Targeted Triphenylphosphonium-Based Compounds: Syntheses, Mechanisms of Action, and Therapeutic and Diagnostic Applications. Chemical reviews 117, 10043-10120 (2017). https://doi.org:10.1021/acs.chemrev.7b00042.
13. Kalyanaraman, B. et al. A review of the basics of mitochondrial bioenergetics, metabolism, and related signaling pathways in cancer cells: Therapeutic targeting of tumor mitochondria with lipophilic cationic compounds. Redox Biol 14, 316-327 (2018). https://doi.org:10.1016/j.redox.2017.09.020
14. Murphy, J. W. et al. Structural and functional basis of CXCL12 (stromal cell-derived factor-1 alpha) binding to heparin. The Journal of biological chemistry 282, 10018-10027 (2007). https://doi.org:10.1074/jbc.M608796200
15. Ross, M. F. et al. Rapid and extensive uptake and activation of hydrophobic triphenylphosphonium cations within cells. Biochem J. 411, 633-645. doi: 610.1042/BJ20080063. (2008).
16. Hanahan, D. & Weinberg, R. A. Hallmarks of cancer: the next generation. Cell 144, 646-674 (2011). https://doi.org:10.1016/j.cell.2011.02.013.
17. Murphy, M. P. Selective targeting of bioactive compounds to mitochondria. Trends Biotechnol. 15, 326-330. doi: 310.1016/50167-7799(1097)01068-01068. (1997).
18. Jauslin, M. L., Meier, T., Smith, R. A. & Murphy, M. P. Mitochondria-targeted antioxidants protect Friedreich Ataxia fibroblasts from endogenous oxidative stress more effectively than untargeted antioxidants. FASEB J. 17, 1972-1974. doi: 1910.1096/fj.1903-0240fje. Epub 23 August 1915. (2003).
19. Cheng, G. et al. Potent inhibition of tumour cell proliferation and immunoregulatory function by mitochondria-targeted atovaquone. Sci Rep. 10, 17872. doi: 17810.11038/s41598-17020-74808-17870. (2020).
20. AbuEid, M. et al. Synchronous effects of targeted mitochondrial complex I inhibitors on tumor and immune cells abrogate melanoma progression. iScience. 24, 102653. doi: 102610.101016/j.isci.102021.102653. eCollection 102021 June 102625. (2021).
21. Kulkarni, C. A. et al. A Novel Triphenylphosphonium Carrier to Target Mitochondria without Uncoupling Oxidative Phosphorylation. J Med Chem. 64, 662-676. doi: 610.1021/acs.jmedchem.1020c01671. Epub 0221 January 01674. (2021).
22. Meanwell, N. A. Fluorine and Fluorinated Motifs in the Design and Application of Bioisosteres for Drug Design. J Med Chem. 61, 5822-5880. doi: 5810.1021/acs.jmedchem.5827b01788. Epub 02018 February 01785. (2018).
23. Alvarez, S. A cartography of the van der Waals territories. Dalton Trans. 42, 8617-8636. doi: 8610.1039/c8613dt50599e. Epub 52013 May 50591. (2013).
24. Inoue, M., Sumii, Y. & Shibata, N. Contribution of Organofluorine Compounds to Pharmaceuticals. ACS Omega. 5, 10633-10640. doi: 10610.11021/acsomega.10630c00830. eCollection 12020 May 10619. (2020).
25. Wilson, D. F. Oxidative phosphorylation: regulation and role in cellular and tissue metabolism. J Physiol. 595, 7023-7038. doi: 7010.1113/JP273839. Epub 27217 October 273829. (2017).
26. Plitzko, B. & Loesgen, S. Measurement of Oxygen Consumption Rate (OCR) and Extracellular Acidification Rate (ECAR) in Culture Cells for Assessment of the Energy Metabolism. Bio Protoc. 8, e2850. doi: 2810.21769/BioProtoc.22850. eCollection 22018 May 21720. (2018).
27. Ehinger, J. K. et al. Cell-permeable succinate prodrugs bypass mitochondrial complex I deficiency. Nat Commun. 7:12317., 10.1038/ncomms12317. (2016).
28. Piel, S. et al. Bioenergetic bypass using cell-permeable succinate, but not methylene blue, attenuates metformin-induced lactate production. Intensive Care Med Exp. 6, 22. doi: 10.1186/s40635-40018-40186-40631. (2018).
29. Callahan, J. & Kopecek, J. Semitelechelic HPMA copolymers functionalized with triphenylphosphonium as drug carriers for membrane transduction and mitochondrial localization. Biomacromolecules. 7, 2347-2356. doi: 2310.1021/bm060336m. (2006).
30. Kelso, G. F. et al. Selective targeting of a redox-active ubiquinone to mitochondria within cells: antioxidant and antiapoptotic properties. J Biol Chem. 276, 4588-4596. doi: 4510.1074/jbc.M009093200. Epub 0090920-11-009093222. (2001).
31. Scaduto, R. C., Jr. & Grotyohann, L. W. Measurement of mitochondrial membrane potential using fluorescent rhodamine derivatives. Biophys J. 76, 469-477. doi: 410.1016/50006-3495(1099)77214-77210. (1999).
32. Bernas, T. & Dobrucki, J. Mitochondrial and nonmitochondrial reduction of MTT: interaction of MTT with TMRE, JC-1, and NAO mitochondrial fluorescent probes. Cytometry. 47, 236-242. doi: 210.1002/cyto.10080. (2002).
33. Seyfried, T. N., Arismendi-Morillo, G., Mukherjee, P. & Chinopoulos, C. On the Origin of ATP Synthesis in Cancer. iScience. 23, 101761. doi: 101710.101016/j.isci.102020.101761. eCollection 10220 November 101720. (2020).
34. Herzig, S. & Shaw, R. J. AMPK: guardian of metabolism and mitochondrial homeostasis. Nat Rev Mol Cell Biol. 19, 121-135. doi: 110.1038/nrm.2017.1095. Epub 2017 October 1034. (2018).
35. Thuronyi, B. W., Privalsky, T. M. & Chang, M. C. Y. Engineered Fluorine Metabolism and Fluoropolymer Production in Living Cells. Angew Chem Int Ed Engl. 56, 13637-13640. doi: 13610.11002/anie.201706696. Epub 20170217 September 201706626. (2017).
36. Buskiewicz, I. A. et al. Reactive oxygen species induce virus-independent MAVS oligomerization in systemic lupus erythematosus. Sci Signal. 9, ra115. doi: 110.1126/scisignal.aaf1933. (2016).
37. Cheng, G. et al. Antiproliferative effects of mitochondria-targeted cationic antioxidants and analogs: Role of mitochondrial bioenergetics and energy-sensing mechanism. Cancer Lett 365, 96-106 (2015). https://doi.org:10.1016/j.canlet.2015.05.016
38. Rodriguez-Gasc6n, A., Solinis, M. & Isla, A. The Role of PK/PD Analysis in the Development and Evaluation of Antimicrobials. Pharmaceutics. 13, 833. doi: 810.3390/pharmaceutics13060833. (2021).
39. McCreight, L. J., Bailey, C. J. & Pearson, E. R. Metformin and the gastrointestinal tract. Diabetologia. 59, 426-435. doi: 410.1007/s00125-00015-03844-00129. Epub 0216 January 00116. (2016).
40. Graham, G. G. et al. Clinical pharmacokinetics of metformin. Clin Pharmacokinet. 50, 81-98. doi: 10.2165/11534750-000000000-000000000. (2011).
41. Smith, R. A. & Murphy, M. P. Animal and human studies with the mitochondria-targeted antioxidant MitoQ. Ann N Y Acad Sci. 1201:96-103., 10.1111/j.1749-6632.2010.05627.x.(2010).
42. Herrera, D., Peral, D. & Bayón, J. C. Preparation of carboxylic arylphosphines by hydrolysis of the trifluoromethyl group. RSC Adv. 12, 7103-7114. doi: 7110.1039/d7102ra00420h. eCollection 02022 March 00421. (2022).
43. Fortun, S. & Schmitzer, A. R. Synthesis and Characterization of Biguanide and Biguanidium Surfactants for Efficient and Recyclable Application in the Suzuki-Miyaura Reaction. ACS Omega. 3, 1889-1896. doi: 1810.1021/acsomega.1887b01962. eCollection 02018 February 01928. (2018).

1. A compound of formula (I′), 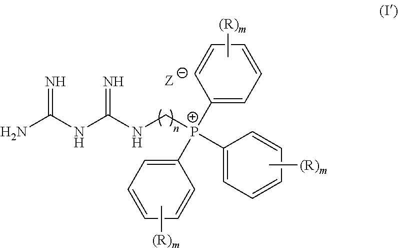
wherein
R at each occurrence is independently F, Cl, CH₂X, CHX₂, CX₃, or OCH₃;
m at each occurrence is independently 1, 2, 3, 4, or 5;
n is an integer from 1-20;
X at each occurrence is independently halogen; and
Z is a counterion.

2. The compound of claim 1, which is a compound of formula (I) 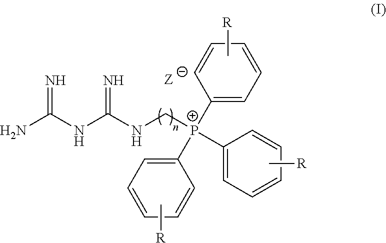
wherein
R is F, Cl, CH₂X, CHX₂, or CX₃; and
X is F or Cl.

3. The compound of claim 1, wherein R is CX₃.

4. The compound of claim 1, wherein X is F.

5. The compound of claim 1, wherein Z is Br.

6. The compound of claim 1, wherein the compound is

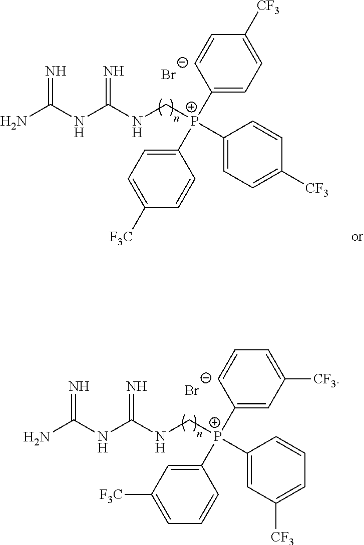

7. The compound of claim 1, wherein n is 10.

8. The compound of claim 1, wherein the compound is

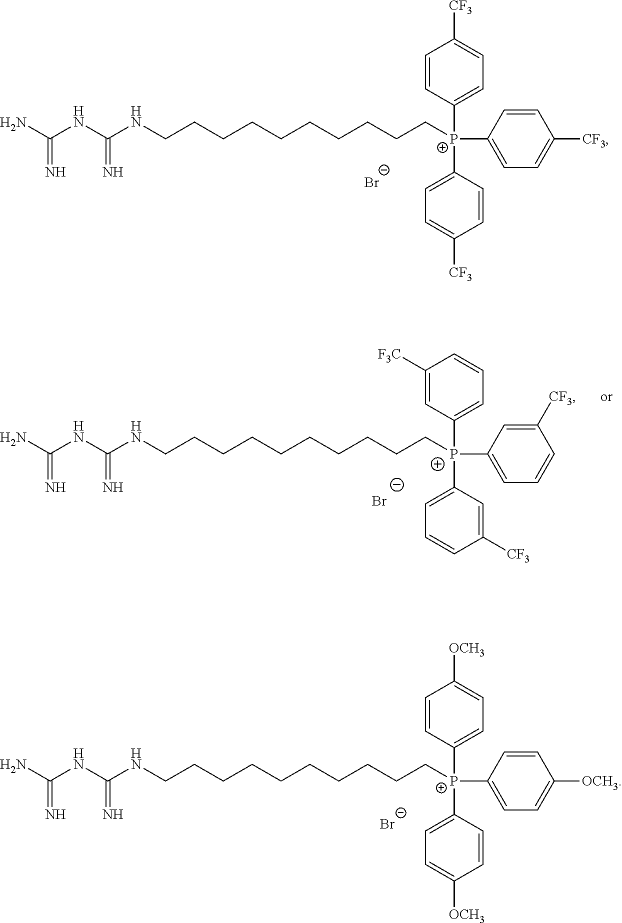

9. The compound of claim 1, wherein the compound is

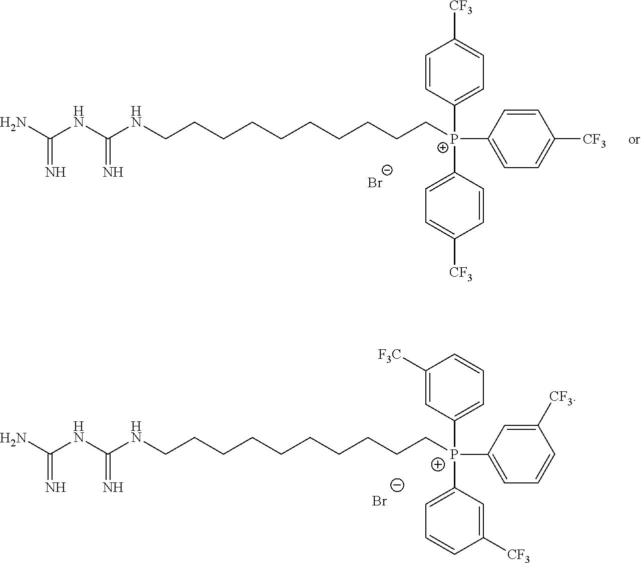 [SMILES: N=C(N)NC(=N)NCCCCCCCCCC[P+](c1ccc(C(F)(F)F)cc1)(c1ccc(C(F)(F)F)cc1)c1ccc(C(F)(F)F)cc1 ; [Br-] ; "or" ; N=C(N)NC(=N)NCCCCCCCCCC[P+](c1cccc(C(F)(F)F)c1)(c1cccc(C(F)(F)F)c1)c1cccc(C(F)(F)F)c1 ; [Br-]]

10. A pharmaceutical composition comprising the compound claim 1 and a pharmaceutically acceptable carrier, diluent, or excipient.

11. A method of treating cancer in a subject in need thereof, wherein the method comprises administering a therapeutically effective amount of the compound of claim 1 to the subject in order to treat the cancer.

12. The method of claim 11, wherein the compound has a reduced toxicity towards a non-transformed cell, as compared with

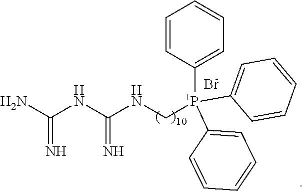

13. The method of claim 11, wherein the compound has a higher selectivity towards a cancer cell than a non-transformed cell, as compared with

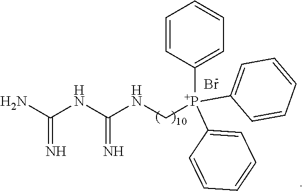

14. The method of claim 11, wherein the compound selectively inhibits mitochondrial energy metabolism in a cancer cell, relative to a non-transformed cell.

15. A method of reducing or inhibiting cancer cell growth in a subject in need thereof, the method comprising administering a therapeutically effective amount of the compound of claim 1 to the subject to reduce or inhibit cell growth.

16. The method of claim 11, wherein the compound is administered orally.

17. A method of inhibiting, reducing or treating metastasis of a cancer in a subject in need thereof, the method comprising administering to the subject a therapeutically effective amount of the compound of claim 1 to the subject in need thereof to inhibit, reduce or treat the metastasis.

18. Use of a compound according to claim 1 for the manufacture of a medicament for the treatment of cancer.

19. The method of claim 11, wherein the cancer is breast cancer, colon cancer, gastric cancer, kidney cancer, liver cancer, lung cancer, ovarian cancer, pancreatic cancer, prostate cancer, non-Hodgkin's lymphoma, leukemia, or a combination thereof.

20. A kit comprising the pharmaceutical composition of claim 10 and instructional material.
